(19) United States
(12) Patent Application Publication
(10) Pub. No.: US 2012/0244188 A1
(43) Pub. Date: Sep. 27, 2012

(54) Treatment of Sensory Disturbance Disorders

(75) Inventors: Andrew M. Blumenfeld, Del Mar, CA (US); Katherine Cernok, Costa Mesa, CA (US); Mitchell F. Brin, Newport Beach, CA (US)

(73) Assignee: ALLERGAN, INC., Irvine, CA (US)

(21) Appl. No.: 13/425,053

(22) Filed: Mar. 20, 2012

Related U.S. Application Data
(60) Provisional application No. 61/467,800, filed on Mar. 25, 2011.
(60) Provisional application No. 61/468,218, filed on Mar. 28, 2011.
(60) Provisional application No. 61/468,940, filed on Mar. 29, 2011.
(60) Provisional application No. 61/468,977, filed on Mar. 29, 2011.

(51) Int. Cl. A61K 38/48; A61P 27/00; A61P 1/12; A61P 1/00; A61P 27/16; A61P 27/02; A61K 39/08; A61P 1/08

(52) U.S. Cl. 424/239.1; 424/94.63

(57) ABSTRACT

The present specification discloses methods of treating a sensory disturbance disorder in an individual using Targeted Endopeptidase Modulators (TEMs), compositions comprising such TEMs, compositions comprising such TEMs and Clostridial toxins, use of such TEMs and/or Clostridial toxins in manufacturing a medicament for treating a sensory disturbance disorder, use of such TEMs and Clostridial toxins in manufacturing a medicament for treating a sensory disturbance disorder, use of such TEMs in treating a sensory disturbance disorder, and use of such TEMs and Clostridial toxins in treating a sensory disturbance disorder.

This application claims the benefit of priority pursuant to 35 U.S.C. §119(e) to U.S. provisional patent application Ser. No. 61/467,800, filed Mar. 25, 2011; Ser. No. 61/468,218, filed Mar. 28, 2011; Ser. No. 61/468,940, filed Mar. 29, 2011; Ser. No. 61/468,977, filed Mar. 29, 2011, each incorporated entirely by reference.

The ability of Clostridial toxins, such as, e.g., Botulinum neurotoxins (BoNTs), BoNT/A, BoNT/B, BoNT/C1, BoNT/D, BoNT/E, BoNT/F and BoNT/G to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Clostridial toxin therapies have been successfully used for many indications. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, a individual administered a therapeutically effective amount of a BoNT/A treatment into the neck muscles for cervical dystonia may develop dysphagia because of dispersal of the toxin into the oropharynx. As another example, a individual administered a therapeutically effective amount of a BoNT/A treatment into the bladder for overactive bladder may develop dry mouth and/or dry eyes. Thus, there still remains a need for treatments having the therapeutic effects that only larger doses of a Clostridial toxin can currently provide, but reduce or prevent the undesirable side-effects associated with larger doses of a Clostridial toxin administration.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat sensory, sympathetic, and/or parasympathetic nerve-based ailments, such as, e.g., various kinds of sensory disturbance disorders. One approach that is currently being exploited involves modifying a Clostridial toxin such that the modified toxin has an altered cell targeting capability for a neuronal or non-neuronal cell of interest. Called re-targeted endopeptidases or Targeted Vesicular Exocytosis Modulator Proteins (TVEMPs) or Targeted Exocytosis Modulators (TEMs), these molecules achieve their exocytosis inhibitory effects by targeting a receptor present on the neuronal or non-neuronal target cell of interest. This re-targeted capability is achieved by replacing the naturally-occurring binding domain of a Clostridial toxin with a targeting domain showing a selective binding activity for a non-Clostridial toxin receptor present in a cell of interest. Such modifications to the binding domain result in a molecule that is able to selectively bind to a non-Clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

The present specification discloses TEMs, compositions comprising TEMs, and methods for treating an individual suffering from a sensory disturbance disorder. This is accomplished by administering a therapeutically effective amount of a composition comprising a TEM to an individual in need thereof. The disclosed methods provide a safe, inexpensive, outpatient-based treatment for the treatment of involuntary movement disorders. In addition, the therapies disclosed herein reduce or prevent unwanted side-effects associated with larger Clostridial toxin doses. These and related advantages are useful for various clinical applications, such as, e.g., the treatment of sensory disturbance disorders where a larger amount of a Clostridial toxin to an individual could produce a beneficial effect, but for the undesirable side-effects.

### SUMMARY

With reference to sensory disturbance disorders as disclosed herein, and without wishing to be limited by any particular theory, it is believed that sympathetic, parasympathetic, and/or sensory neurons have important functions in aspects of sensory perception and that improper innervations from these types of neurons can contribute to one or more different types of sensory disturbance disorders. As such, TEMs comprising a targeting domain for a receptor present on sympathetic, parasympathetic, and/or sensory neurons can reduce or prevent these improper innervations, thereby reducing or preventing one or more symptoms associate with a sensory disturbance disorder. It is further theorized that such a TEM in combination with a Clostridial toxin can provide enhanced, if not synergistic, therapeutic benefit because such a combination also inhibit motor neurons. However, using a combination therapy of such a TEM with a Clostridial toxin, also allows a lower dose of a Clostridial toxin to be administered to treat a sensory disturbance disorder. This will result in a decrease in muscle weakness generated in the compensatory muscles relative to the current treatment paradigm. As such, a combined therapy using a Clostridial toxin and a TEM comprising a targeting domain for a receptor present on sympathetic, parasympathetic, and/or sensory neurons can reduce or prevent these improper innervations, and in combination can reduce or prevent one or more symptoms associate with a sensory disturbance disorder.

Thus, aspects of the present specification disclose methods of treating a sensory disturbance disorder in an individual, the methods comprising the step of administering to the individual in need thereof a therapeutically effective amount of a composition including a TEM, wherein administration of the composition reduces a symptom of the sensory disturbance disorder, thereby treating the individual. In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. A sensory disturbance disorder includes, without limitation, a sensory processing disorder, an auditory disturbance, an olfactory disturbance, a gustatory disturbance, a visual disturbance, a hallucination disorder, a nausea, a vomiting, and a gastrointestinal dysfunction.

Other aspects of the present specification disclose uses of a TEM disclosed herein in the manufacturing a medicament for treating a sensory disturbance disorder disclosed herein in an individual in need thereof.

Yet other aspects of the present specification uses of a TEM disclosed herein in the treatment of a sensory disturbance disorder disclosed herein in an individual in need thereof.

Other aspects of the present specification disclose methods of treating a sensory disturbance disorder in an individual, the methods comprising the step of administering to the individual in need thereof a therapeutically effective amount of a composition including a Clostridial neurotoxin and a TEM, wherein administration of the composition reduces a symptom of the sensory disturbance, thereby treating the individual. A Clostridial neurotoxin includes, without limitation, a Botulinum toxin (BoNT), a Tetanus toxin (TeNT), a Baratii toxin (BaNT), and a Butyricum toxin (BuNT). In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. A sensory disturbance disorder includes, without limitation, a sensory processing disorder, an auditory disturbance, an olfactory disturbance, a gustatory disturbance, a visual disturbance, a hallucination disorder, a nausea, a vomiting, and a gastrointestinal dysfunction.

Other aspects of the present specification disclose uses of a Clostridial neurotoxin and a TEM disclosed herein in the manufacturing a medicament for treating a sensory disturbance disorder disclosed herein in an individual in need thereof.

Yet other aspects of the present specification uses of a Clostridial neurotoxin and a TEM disclosed herein in the treatment of a sensory disturbance disorder disclosed herein in an individual in need thereof.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the HN domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a retargeted peptide binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the H_{CC} region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TEM domain organization with a targeting domain located at the amino terminus of a TEM. FIG. 3A depicts the single-chain polypeptide form of a TEM with an amino to carboxyl linear organization comprising a targeting domain, a translocation domain, a di-chain loop region comprising an exogenous protease cleavage site (P), and an enzymatic domain. Upon proteolytic cleavage with a P protease, the single-chain form of the TEM is converted to the di-chain form. FIG. 3B depicts the single polypeptide form of a TEM with an amino to carboxyl linear organization comprising a targeting domain, an enzymatic domain, a di-chain loop region comprising an exogenous protease cleavage site (P), and a translocation domain. Upon proteolytic cleavage with a P protease, the single-chain form of the TEM is converted to the di-chain form.

FIG. 4 shows a TEM domain organization with a targeting domain located between the other two domains. FIG. 4A depicts the single polypeptide form of a TEM with an amino to carboxyl linear organization comprising an enzymatic domain, a di-chain loop region comprising an exogenous protease cleavage site (P), a targeting domain, and a translocation domain. Upon proteolytic cleavage with a P protease, the single-chain form of the TEM is converted to the di-chain form. FIG. 4B depicts the single polypeptide form of a TEM with an amino to carboxyl linear organization comprising a translocation domain, a di-chain loop region comprising an exogenous protease cleavage site (P), a targeting domain, and an enzymatic domain. Upon proteolytic cleavage with a P protease, the single-chain form of the TEM is converted to the di-chain form. FIG. 4C depicts the single polypeptide form of a TEM with an amino to carboxyl linear organization comprising an enzymatic domain, a targeting domain, a di-chain loop region comprising an exogenous protease cleavage site (P), and a translocation domain. Upon proteolytic cleavage with a P protease, the single-chain form of the TEM is converted to the di-chain form. FIG. 4D depicts the single polypeptide form of a TEM with an amino to carboxyl linear organization comprising a translocation domain, a targeting domain, a di-chain loop region comprising an exogenous protease cleavage site (P), and an enzymatic domain. Upon proteolytic cleavage with a P protease, the single-chain form of the TEM is converted to the di-chain form.

FIG. 5 shows a TEM domain organization with a targeting domain located at the carboxyl terminus of the TEM. FIG. 5A depicts the single polypeptide form of a TEM with an amino to carboxyl linear organization comprising an enzymatic domain, a di-chain loop region comprising an exogenous protease cleavage site (P), a translocation domain, and a targeting domain. Upon proteolytic cleavage with a P protease, the single-chain form of the TEM is converted to the di-chain form. FIG. 5B depicts the single polypeptide form of a TEM with an amino to carboxyl linear organization comprising a translocation domain, a di-chain loop region comprising an exogenous protease cleavage site (P), an enzymatic domain, and a targeting domain. Upon proteolytic cleavage with a P protease, the single-chain form of the TEM is converted to the di-chain form.

### DESCRIPTION

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, BoNT/B, BoNT/E and BoNT/F), animals (BoNT/C1 and BoNT/D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently five BoNT/A subtypes, BoNT/A1, BoNT/A2, BoNT/A3 BoNT/A4 and BoNT/A5, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other Clostridia species, C. baratii and C. butyricum, produce toxins, BaNT and BuNT, which are functionally similar to BoNT/F and BoNT/E, respectively.

Clostridial toxins are released by Clostridial bacterium as complexes comprising the approximately 150-kDa Clostridial toxin along with associated non-toxin proteins (NAPs). Identified NAPs include proteins possessing hemagglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa. Thus, the botulinum toxin type A complex can be produced by Clostridial bacterium as 900-kDa, 500-kDa and 300-kDa forms. Botulinum toxin types B and C₁ are apparently produced as only a 500-kDa complex. Botulinum toxin type D is produced as both 300-kDa and 500-kDa complexes. Finally, botulinum toxin types E and F are produced as only approximately 300-kDa complexes. The differences in molecular weight for the complexes are due to differing ratios of NAPs. The toxin complex is important for the intoxication process because it provides protection from adverse environmental conditions, resistance to protease digestion, and appears to facilitate internalization and activation of the toxin.

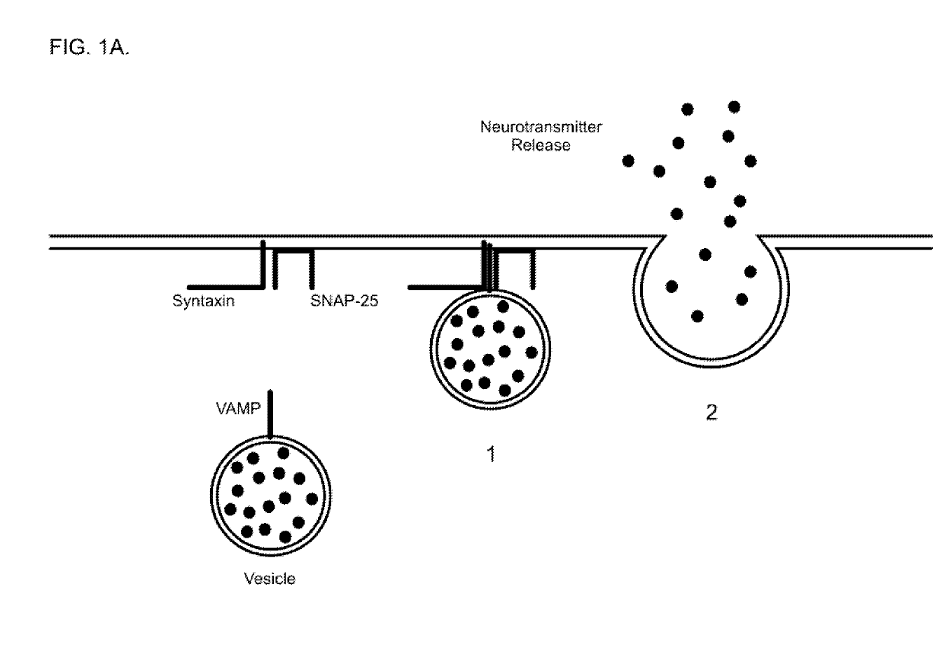

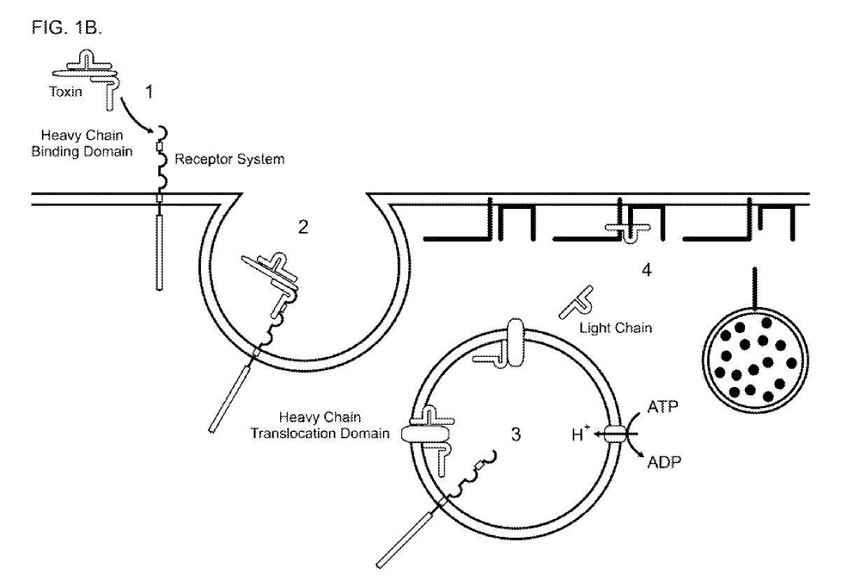

A Clostridial toxin itself is translated as a single chain polypeptide that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule of a Clostridial toxin comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain contained within the amino-terminal half of the heavy chain (H_{N}) that facilitates release of the LC from intracellular vesicles into the cytoplasm of the target cell; and 3) a binding domain found within the carboxyl-terminal half of the heavy chain (H_{C}) that determines the binding activity and binding specificity of the toxin to the receptor complex located at the surface of the target cell. The H_{C} domain comprises two distinct structural features of roughly equal size that indicate function and are designated the H_{CN} and H_{CC} subdomains.

Clostridial toxins act on the nervous system by blocking the release of acetylcholine (ACh) at the pre-synaptic neuromuscular junction. The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although applicants have no wish to be limited by the following description, the intoxication mechanism can be described as comprising at least four steps: 1) receptor binding, 2) complex internalization, 3) light chain translocation, and 4) enzymatic target modification (FIG. 1). The process is initiated when the binding domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate pH-dependent structural rearrangements that increase hydrophobicity, create a pore in the vesicle membrane, and promote formation of the di-chain form of the toxin. Once di-chain formation occurs, light chain endopeptidase of the toxin is released from the intracellular vesicle via the pore into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface.

Aspects of the present specification disclose, in part, in part, a Clostridial toxin. As used herein, the term “Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof.

A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

Clostridial toxins can be produced using standard purification or recombinant biology techniques known to those skilled in the art. See, e.g., Hui Xiang et al., Animal Product Free System and Process for Purifying a Botulinum Toxin, U.S. Pat. No. 7,354,740, which is hereby incorporated by reference in its entirety. For example, a BoNT/A complex can be isolated and purified from an anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline BoNT/A complex with a specific potency of 3×10⁷ LD₅₀ U/mg or greater. Furthermore, NAPs can be separated out to obtain purified toxin, such as e.g., BoNT/A with an approximately 150 kD molecular weight with a specific potency of 1-2×10⁸ LD₅₀ U/mg or greater, purified BoNT/B with an approximately 156 kD molecular weight with a specific potency of 1-2×10⁸ LD₅₀ U/mg or greater, and purified BoNT/F with an approximately 155 kD molecular weight with a specific potency of 1-2×10⁷ LD₅₀ U/mg or greater. See Edward J. Schantz & Eric A. Johnson, Properties and use of Botulinum Toxin and Other Microbial Neurotoxins in Medicine, Microbiol Rev. 56: 80-99 (1992), which is hereby incorporated in its entirety. As another example, recombinant Clostridial toxins can be recombinantly produced as described in Steward et al., Optimizing Expression of Active Botulinum Toxin Type A, U.S. Patent Publication 2008/0057575; and Steward et al., Optimizing Expression of Active Botulinum Toxin Type E, U.S. Patent Publication 2008/0138893, each of which is hereby incorporated in its entirety.

Clostridial toxins are also commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). Clostridial toxin complexes may be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

In an embodiment, a Clostridial may be a Botulinum toxin, Tetanus toxin, a Baratii toxin, or a Butyricum toxin. In aspects of this embodiment, a Botulinum toxin may be a BoNT/A, a BoNT/B, a BoNT/C₁, a BoNT/D, a BoNT/E, a BoNT/F, or a BoNT/G. In another embodiment, a Clostridial toxin may be a Clostridial toxin variant. In aspects of this embodiment, a Clostridial toxin variant may be a naturally-occurring Clostridial toxin variant or a non-naturally-occurring Clostridial toxin variant. In other aspects of this embodiment, a Clostridial toxin variant may be a BoNT/A variant, a BoNT/B variant, a BoNT/C₁ variant, a BoNT/D variant, a BoNT/E variant, a BoNT/F variant, a BoNT/G variant, a TeNT variant, a BaNT variant, or a BuNT variant, where the variant is either a naturally-occurring variant or a non-naturally-occurring variant.

In an embodiment, a Clostridial toxin may be a Clostridial toxin complex. In aspects of this embodiment, a Clostridial toxin complex may be a BoNT/A complex, a BoNT/B complex, a BoNT/C₁ complex, a BoNT/D complex, a BoNT/E complex, a BoNT/F complex, a BoNT/G complex, a TeNT complex, a BaNT complex, or a BuNT complex. In other aspects of this embodiment, a Clostridial toxin complex may be a 900-kDa BoNT/A complex, a 500-kDa BoNT/A complex, a 300-kDa BoNT/A complex, a 500-kDa BoNT/B complex, a 500-kDa BoNT/C1 complex, a 500-kDa BoNT/D complex, a 300-kDa BoNT/D complex, a 300-kDa BoNT/E complex, or a 300-kDa BoNT/F complex.

Aspects of the present disclosure comprise, in part, a Targeted Exocytosis Modulator. As used herein, the term “Targeted Exocytosis Modulator” is synonymous with “TEM” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

However, an important difference between TEMs, such as, e.g., TEMs disclosed herein, and native Clostridial toxins is that since TEMs do not target motor neurons, the lethality associated with over-dosing an individual with a TEM is greatly minimized, if not avoided altogether. For example, a TEM comprising an opioid targeting domain can be administered at 10,000 times the therapeutically effective dose before evidence of lethality is observed, and this lethality is due to the passive diffusion of the molecule and not via the intoxication process. Thus, for all practical purposes TEMs are non-lethal molecules.

As used herein, the term “Clostridial toxin enzymatic domain” refers to a Clostridial toxin polypeptide located in the light chain of a Clostridial toxin that executes the enzymatic target modification step of the intoxication process. A Clostridial toxin enzymatic domain includes a metalloprotease region containing a zinc-dependent endopeptidase activity which specifically targets core components of the neurotransmitter release apparatus. Thus, a Clostridial toxin enzymatic domain specifically targets and proteolytically cleavages of a Clostridial toxin substrate, such as, e.g., SNARE proteins like a SNAP-25 substrate, a VAMP substrate and a Syntaxin substrate.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimeras, active Clostridial toxin enzymatic domain fragments thereof, or any combination thereof. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

As used herein, the term “Clostridial toxin translocation domain” refers to a Clostridial toxin polypeptide located within the amino-terminal half of the heavy chain of a Clostridial toxin that executes the translocation step of the intoxication process. The translocation step appears to involve an allosteric conformational change of the translocation domain caused by a decrease in pH within the intracellular vesicle. This conformational change results in the formation of a pore in the vesicular membrane that permits the movement of the light chain from within the vesicle into the cytoplasm. Thus, a Clostridial toxin translocation domain facilitates the movement of a Clostridial toxin light chain across a membrane of an intracellular vesicle into the cytoplasm of a cell.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

As used herein, the term “targeting domain” is synonymous with “binding domain” or “targeting moiety” and refers to a polypeptide that executes the receptor binding and/or complex internalization steps of the intoxication process, with the proviso that the binding domain is not a Clostridial toxin binding domain found within the carboxyl-terminal half of the heavy chain of a Clostridial toxin. A targeting domain includes a receptor binding region that confers the binding activity and/or specificity of the targeting domain for its cognate receptor. As used herein, the term “cognate receptor” refers to a receptor for which the targeting domain preferentially interacts with under physiological conditions, or under in vitro conditions substantially approximating physiological conditions. As used herein, the term “preferentially interacts” is synonymous with “preferentially binding” and refers to an interaction that is statistically significantly greater in degree relative to a control. With reference to a targeting domain disclosed herein, a targeting domain binds to its cognate receptor to a statistically significantly greater degree relative to a non-cognate receptor. Said another way, there is a discriminatory binding of the targeting domain to its cognate receptor relative to a non-cognate receptor. Thus, a targeting domain directs binding to a TEM-specific receptor located on the plasma membrane surface of a target cell.

In an embodiment, a targeting domain disclosed herein has an association rate constant that confers preferential binding to its cognate receptor. In aspects of this embodiment, a targeting domain disclosed herein binds to its cognate receptor with an association rate constant of, e.g., less than 1×10⁵ M⁻¹ s⁻¹, less than 1×10⁶ M⁻¹ s⁻¹, less than 1×10⁷ M⁻¹ s⁻¹, or less than 1×10⁸ M⁻¹ s⁻¹. In other aspects of this embodiment, a targeting domain disclosed herein binds to its cognate receptor with an association rate constant of, e.g., more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹. In yet other aspects of this embodiment, a targeting domain disclosed herein binds to its cognate receptor with an association rate constant between 1×10⁵ M⁻¹ s⁻¹ to 1×10⁸ M⁻¹ s⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In another embodiment, a targeting domain disclosed herein has an association rate constant that is greater for its cognate target receptor relative to a non-cognate receptor. In other aspects of this embodiment, a targeting domain disclosed herein has an association rate constant that is greater for its cognate target receptor relative to a non-cognate receptor by, at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an association rate constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In yet another embodiment, a targeting domain disclosed herein has a disassociation rate constant that confers preferential binding to its cognate receptor. In other aspects of this embodiment, a targeting domain disclosed herein binds to its cognate receptor with a disassociation rate constant of less than 1×10⁻³ s⁻¹, less than 1×10⁻⁴ s⁻¹, or less than 1×10⁻⁵ s⁻¹. In yet other aspects of this embodiment, a targeting domain disclosed herein binds to its cognate receptor with a disassociation rate constant of, e.g., less than 1.0×10⁻⁴ s⁻¹, less than 2.0×10⁻⁴ s⁻¹, less than 3.0×10⁻⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In still other aspects of this embodiment, a targeting domain disclosed herein binds to its cognate receptor with a disassociation rate constant of, e.g., more than 1×10⁻³ s⁻¹, more than 1×10⁻⁴ s⁻¹, or more than 1×10⁻⁵ s⁻¹. In other aspects of this embodiment, a targeting domain disclosed herein binds to its cognate receptor with a disassociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 9.0×10⁻⁴ s⁻¹.

In still another embodiment, a targeting domain disclosed herein has a disassociation rate constant that is less for its cognate target receptor relative to a non-cognate receptor. In other aspects of this embodiment, a targeting domain disclosed herein has a disassociation rate constant that is less for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has a disassociation rate constant that is less for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In another embodiment, a targeting domain disclosed herein has an equilibrium disassociation constant that confers preferential binding to its cognate receptor. In other aspects of this embodiment, a targeting domain disclosed herein binds to its cognate receptor with an equilibrium disassociation constant of, e.g., less than 0.500 nM. In yet other aspects of this embodiment, a targeting domain disclosed herein binds to its cognate receptor with an equilibrium disassociation constant of, e.g., less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM. In other aspects of this embodiment, a targeting domain disclosed herein binds to its cognate receptor with an equilibrium disassociation constant of, e.g., more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.

In yet another embodiment, a targeting domain disclosed herein has an equilibrium disassociation constant that is greater for its cognate target receptor relative to a non-cognate receptor. In other aspects of this embodiment, a targeting domain disclosed herein has an equilibrium disassociation constant that is greater for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an equilibrium disassociation constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In another embodiment, a targeting domain disclosed herein may be one that preferentially interacts with a receptor located on a sensory neuron. In an aspect of this embodiment, the sensory neuron targeting domain is one whose cognate receptor is located exclusively on the plasma membrane of sensory neurons. In another aspect of this embodiment, the sensory neuron targeting domain is one whose cognate receptor is located primarily on the plasma membrane of sensory neuron. For example, a receptor for a sensory neuron targeting domain is located primarily on a sensory neuron when, e.g., at least 60% of all cells that have a cognate receptor for a sensory neuron targeting domain on the surface of the plasma membrane are sensory neurons, at least 70% of all cells that have a cognate receptor for a sensory neuron targeting domain on the surface of the plasma membrane are sensory neurons, at least 80% of all cells that have a cognate receptor for a sensory neuron targeting domain on the surface of the plasma membrane are sensory neurons, or at least 90% of all cells that have a cognate receptor for a sensory neuron targeting domain on the surface of the plasma membrane are sensory neurons. In yet another aspect of this embodiment, the sensory neuron targeting domain is one whose cognate receptor is located on the plasma membrane of several types of cells, including sensory neurons. In still another aspect of this embodiment, the sensory neuron targeting domain is one whose cognate receptor is located on the plasma membrane of several types of cells, including sensory neurons, with the proviso that motor neurons are not one of the other types of cells.

In another embodiment, a targeting domain disclosed herein may be one that preferentially interacts with a receptor located on a sympathetic neuron. In an aspect of this embodiment, the sympathetic neuron targeting domain is one whose cognate receptor is located exclusively on the plasma membrane of sympathetic neurons. In another aspect of this embodiment, the sympathetic neuron targeting domain is one whose cognate receptor is located primarily on the plasma membrane of sympathetic neuron. For example, a receptor for a sympathetic neuron targeting domain is located primarily on a sympathetic neuron when, e.g., at least 60% of all cells that have a cognate receptor for a sympathetic neuron targeting domain on the surface of the plasma membrane are sympathetic neurons, at least 70% of all cells that have a cognate receptor for a sympathetic neuron targeting domain on the surface of the plasma membrane are sympathetic neurons, at least 80% of all cells that have a cognate receptor for a sympathetic neuron targeting domain on the surface of the plasma membrane are sympathetic neurons, or at least 90% of all cells that have a cognate receptor for a sympathetic neuron targeting domain on the surface of the plasma membrane are sympathetic neurons. In yet another aspect of this embodiment, the sympathetic neuron targeting domain is one whose cognate receptor is located on the plasma membrane of several types of cells, including sympathetic neurons. In still another aspect of this embodiment, the sympathetic neuron targeting domain is one whose cognate receptor is located on the plasma membrane of several types of cells, including sympathetic neurons, with the proviso that motor neurons are not one of the other types of cells.

In another embodiment, a targeting domain disclosed herein may be one that preferentially interacts with a receptor located on a parasympathetic neuron. In an aspect of this embodiment, the parasympathetic neuron targeting domain is one whose cognate receptor is located exclusively on the plasma membrane of parasympathetic neurons. In another aspect of this embodiment, the parasympathetic neuron targeting domain is one whose cognate receptor is located primarily on the plasma membrane of parasympathetic neuron. For example, a receptor for a parasympathetic neuron targeting domain is located primarily on a parasympathetic neuron when, e.g., at least 60% of all cells that have a cognate receptor for a parasympathetic neuron targeting domain on the surface of the plasma membrane are parasympathetic neurons, at least 70% of all cells that have a cognate receptor for a parasympathetic neuron targeting domain on the surface of the plasma membrane are parasympathetic neurons, at least 80% of all cells that have a cognate receptor for a parasympathetic neuron targeting domain on the surface of the plasma membrane are parasympathetic neurons, or at least 90% of all cells that have a cognate receptor for a parasympathetic neuron targeting domain on the surface of the plasma membrane are parasympathetic neurons. In yet another aspect of this embodiment, the parasympathetic neuron targeting domain is one whose cognate receptor is located on the plasma membrane of several types of cells, including parasympathetic neurons. In still another aspect of this embodiment, the parasympathetic neuron targeting domain is one whose cognate receptor is located on the plasma membrane of several types of cells, including parasympathetic neurons, with the proviso that motor neurons are not one of the other types of cells.

In another embodiment, a targeting domain disclosed herein is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.

In an aspect of this embodiment, an opioid peptide targeting domain is an enkephalin peptide, a bovine adrenomedullary-22 (BAM22) peptide, an endomorphin peptide, an endorphin peptide, a dynorphin peptide, a nociceptin peptide, or a hemorphin peptide. In another aspect of this embodiment, an enkephalin peptide targeting domain is a Leu-enkephalin peptide, a Met-enkephalin peptide, a Met-enkephalin MRGL peptide, or a Met-enkephalin MRF peptide. In another aspect of this embodiment, a bovine adrenomedullary-22 peptide targeting domain is a BAM22 (1-12) peptide, a BAM22 (6-22) peptide, a BAM22 (8-22) peptide, or a BAM22 (1-22) peptide. In another aspect of this embodiment, an endomorphin peptide targeting domain is an endomorphin-1 peptide or an endomorphin-2 peptide. In another aspect of this embodiment, an endorphin peptide targeting domain an endorphin-α peptide, a neoendorphin-α peptide, an endorphin-β peptide, a neoendorphin-β peptide, or an endorphin-γ peptide. In another aspect of this embodiment, a dynorphin peptide targeting domain is a dynorphin A peptide, a dynorphin B (leumorphin) peptide, or a rimorphin peptide. In another aspect of this embodiment, a nociceptin peptide targeting domain is a nociceptin RK peptide, a nociceptin peptide, a neuropeptide 1 peptide, a neuropeptide 2 peptide, or a neuropeptide 3 peptide. In another aspect of this embodiment, a hemorphin peptide targeting domain is a LVVH7 peptide, a VVH7 peptide, a VH7 peptide, a H7 peptide, a LVVH6 peptide, a LVVH5 peptide, a VVH5 peptide, a LVVH4 peptide, or a LVVH3 peptide.

In an aspect of this embodiment, a galanin peptide targeting domain is a galanin peptide, a galanin message-associated peptide (GMAP) peptide, a galanin like protein (GALP) peptide, or an alarin peptide.

In an aspect of this embodiment, a PAR peptide targeting domain is a PAR1 peptide, a PAR2 peptide, a PAR3 peptide and a PAR4 peptide. In an aspect of this embodiment, a somatostatin peptide targeting domain is a somatostatin peptide or a cortistatin peptide. In an aspect of this embodiment, a neurotensin peptide targeting domain a neurotensin or a neuromedin N. In an aspect of this embodiment, a SLURP peptide targeting domain is a SLURP-1 peptide or a SLURP-2 peptide. In an aspect of this embodiment, an angiotensin peptide targeting domain is an angiotensin peptide.

In an aspect of this embodiment, a tachykinin peptide targeting domain is a Substance P peptide, a neuropeptide K peptide, a neuropeptide gamma peptide, a neurokinin A peptide, a neurokinin B peptide, a hemokinin peptide, or a endokinin peptide. In an aspect of this embodiment, a Neuropeptide Y related peptide targeting domain is a Neuropeptide Y peptide, a Peptide YY peptide, Pancreatic peptide peptide, a Pancreatic icosapeptide peptide, a Pancreatic Hormone domain peptide, a CXCL12 peptide, and a Sjogren syndrome antigen B peptide. In an aspect of this embodiment, a kinin peptide targeting domain is a bradykinin peptide, a kallidin peptide, a desArg9 bradykinin peptide, a desArg10 bradykinin peptide, a kininogen peptide, gonadotropin releasing hormone 1 peptide, chemokine peptide, an arginine vasopressin peptide.

In an aspect of this embodiment, a melanocortin peptide targeting domain comprises a melanocyte stimulating hormone peptide, an adrenocorticotropin peptide, a lipotropin peptide, or a melanocortin peptide derived neuropeptide. In an aspect of this embodiment, a melanocyte stimulating hormone peptide targeting domain comprises an α-melanocyte stimulating hormone peptide, a β-melanocyte stimulating hormone peptide, or a γ-melanocyte stimulating hormone peptide. In an aspect of this embodiment, an adrenocorticotropin peptide targeting domain comprises an adrenocorticotropin or a Corticotropin-like intermediary peptide. In an aspect of this embodiment, a lipotropin peptide targeting domain comprises a β-lipotropin peptide or a γ-lipotropin peptide.

In an aspect of this embodiment, a granin peptide targeting domain comprises a chromogranin A peptide, a chromogranin B peptide, a chromogranin C (secretogranin II) peptide, a secretogranin IV peptide, or a secretogranin VI peptide. In an aspect of this embodiment, a chromogranin A peptide targeting domain comprises a β-granin peptide, a vasostatin peptide, a chromostatin peptide, a pancreastatin peptide, a WE-14 peptide, a catestatin peptide, a parastatin peptide, or a GE-25 peptide. In an aspect of this embodiment, a chromogranin B peptide targeting domain comprises a GAWK peptide, an adrenomedullary peptide, or a secretolytin peptide. In an aspect of this embodiment, a chromogranin C peptide targeting domain comprises a secretoneurin peptide.

In an aspect of this embodiment, a glucagons-like hormone peptide targeting domain is a glucagon-like peptide-1, a glucagon-like peptide-2, a glicentin, a glicentin-related peptide (GRPP), a glucagon, or an oxyntomodulin (OXY). In an aspect of this embodiment, a secretin peptide targeting domain is a secretin peptide. In an aspect of this embodiment, a pituitary adenylate cyclase activating peptide targeting domain is a pituitary adenylate cyclase activating peptide. In an aspect of this embodiment, a growth hormone-releasing hormone peptide targeting domain a growth hormone-releasing hormone peptide. In an aspect of this embodiment, a vasoactive intestinal peptide targeting domain is a vasoactive intestinal peptide-1 peptide or a vasoactive intestinal peptide-2 peptide. In an aspect of this embodiment, a gastric inhibitory peptide targeting domain is a gastric inhibitory peptide. In an aspect of this embodiment, a calcitonin peptide targeting domain is a calcitonin peptide, an amylin peptide, a calcitonin-related peptide α, a calcitonin-related peptide β, and a islet amyloid peptide. In an aspect of this embodiment, a visceral gut peptide targeting domain is a gastrin peptide, a gastrin-releasing peptide, or a cholecystokinin peptide.

In an aspect of this embodiment, a neurotrophin peptide targeting domain is a nerve growth factor (NGF) peptide, a brain derived neurotrophic factor (BDNF) peptide, a neurotrophin-3 (NT-3) peptide, a neurotrophin-4/5 (NT-4/5) peptide, or an amyloid beta (A4) precursor protein neurotrophin (APP) peptide. In an aspect of this embodiment, a head activator peptide targeting domain is a head activator peptide. In an aspect of this embodiment, a glial cell line-derived neurotrophic factor family of ligands peptide targeting domain is a glial cell line-derived neurotrophic factor peptide, a Neurturin peptide, a Persephrin peptide, or an Artemin peptide. In an aspect of this embodiment, a RF-amide related peptide targeting domain a RF-amide related peptide-1, a RF-amide related peptide-2, a RF-amide related peptide-3, a neuropeptide AF, or a neuropeptide FF.

In an aspect of this embodiment, a neurohormone peptide targeting domain is a corticotropin-releasing hormone (CCRH), a parathyroid hormone (PTH), a parathyroid hormone-like hormone (PTHLH), a PHYH, a thyrotropin-releasing hormone (TRH), an urocortin-1 (UCN1), an urocortin-2 (UCN2), an urocortin-3 (UCN3), or an urotensin 2 (UTS2). In an aspect of this embodiment, a neuroregulatory cytokine peptide targeting domain is a ciliary neurotrophic factor peptide, a glycophorin-A peptide, a leukemia inhibitory factor peptide, a cardiotrophin-1 peptide, a cardiotrophin-like cytokine peptide, a neuroleukin peptide, and an onostatin M peptide. In an aspect of this embodiment, an IL peptide targeting domain is an IL-1 peptide, an IL-2 peptide, an IL-3 peptide, an IL-4 peptide, an IL-5 peptide, an IL-6 peptide, an IL-7 peptide, an IL-8 peptide, an IL-9 peptide, an IL-10 peptide, an IL-11 peptide, an IL-12 peptide, an IL-18 peptide, an IL-32 peptide, or an IL-33 peptide.

In an aspect of this embodiment, a VEGF peptide targeting domain is a VEGF-A peptide, a VEGF-B peptide, a VEGF-C peptide, a VEGF-D peptide, or a placenta growth factor (PIGF) peptide. In an aspect of this embodiment, an IGF peptide targeting domain is an IGF-1 peptide or an IGF-2 peptide. In an aspect of this embodiment, an EGF peptide targeting domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4). In an aspect of this embodiment, a FGF peptide targeting domain is a FGF1 peptide, a FGF2 peptide, a FGF3 peptide, a FGF4 peptide, a FGF5 peptide, a FGF6 peptide, a FGF7 peptide, a FGF8 peptide, a FGF9 peptide, a FGF10 peptide, a FGF17 peptide, or a FGF18 peptide. In an aspect of this embodiment, a PDGF peptide targeting domain is a PDGFα peptide or a PDGFβ peptide.

In an aspect of this embodiment, a TGFβ peptide targeting domain is a TGFβ1 peptide, a TGFβ2 peptide, a TGFβ3 peptide, or a TGFβ4 peptide. In an aspect of this embodiment, a BMP peptide targeting domain is a BMP2 peptide, a BMP3 peptide, a BMP4 peptide, a BMP5 peptide, a BMP6 peptide, a BMP7 peptide, a BMP8 peptide, or a BMP10 peptide. In an aspect of this embodiment, a GDF peptide targeting domain is a GDF1 peptide, a GDF2 peptide, a GDF3 peptide, a GDF5 peptide, a GDF6 peptide, a GDF7 peptide, a GDF8 peptide, a GDF10 peptide, a GDF11 peptide, or a GDF15 peptide. In an aspect of this embodiment, an activin peptide targeting domain is an activin A peptide, an activin B peptide, an activin C peptide, an activin E peptide, or an inhibin A peptide.

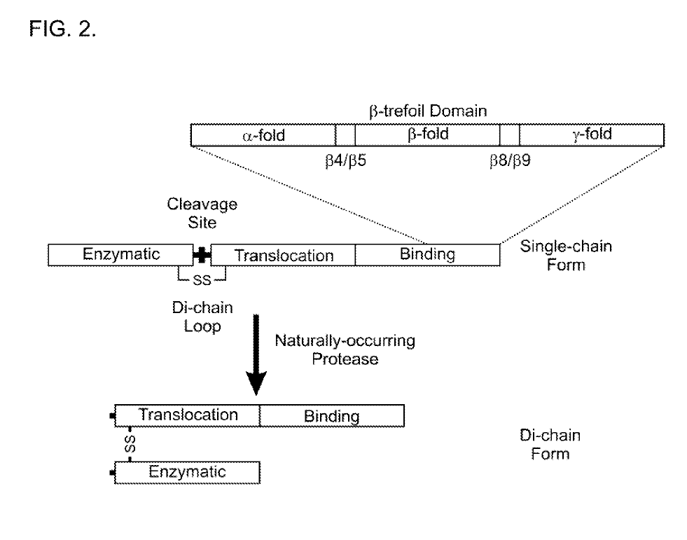

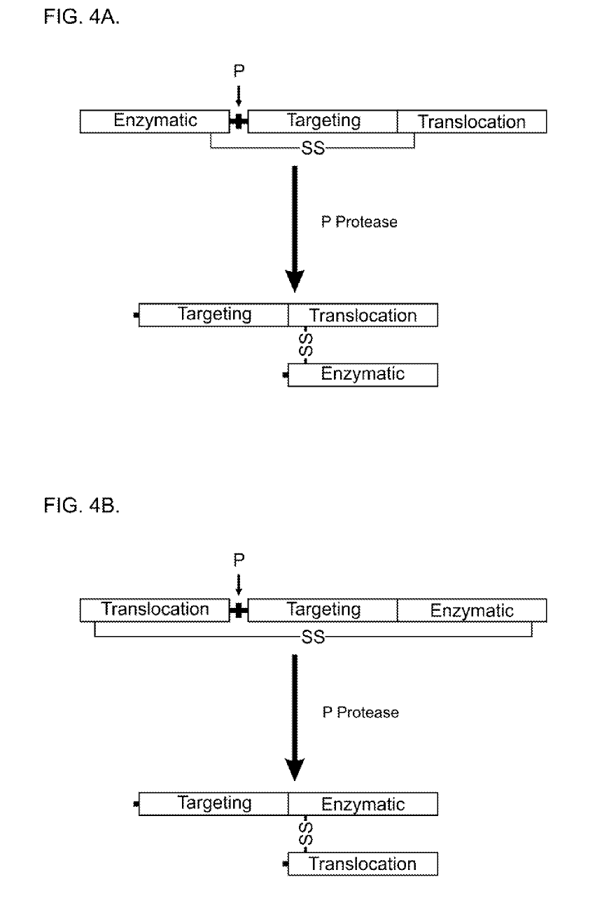

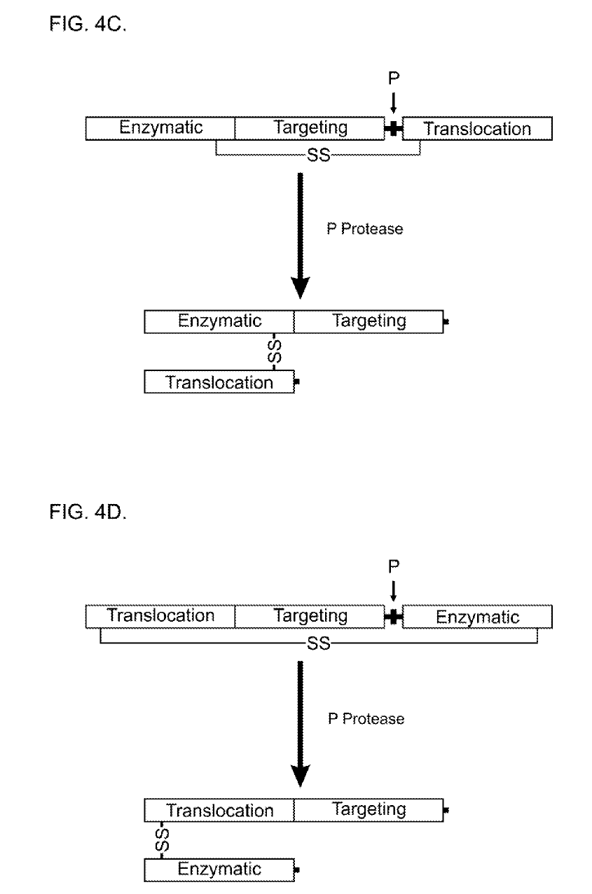

As discussed above, naturally-occurring Clostridial toxins are organized into three functional domains comprising a linear amino-to-carboxyl single polypeptide order of the enzymatic domain (amino region position), the translocation domain (middle region position) and the binding domain (carboxyl region position) (FIG. 2). This naturally-occurring order can be referred to as the carboxyl presentation of the binding domain because the domain necessary for binding to the receptor is located at the carboxyl region position of the Clostridial toxin. However, it has been shown that Clostridial toxins can be modified by rearranging the linear amino-to-carboxyl single polypeptide order of the three major domains and locating a targeting moiety at the amino region position of a Clostridial toxin, referred to as amino presentation, as well as in the middle region position, referred to as central presentation (FIG. 4).

Thus, a TEM can comprise a targeting domain in any and all locations with the proviso that TEM is capable of performing the intoxication process. Non-limiting examples include, locating a targeting domain at the amino terminus of a TEM; locating a targeting domain between a Clostridial toxin enzymatic domain and a Clostridial toxin translocation domain of a TEM; and locating a targeting domain at the carboxyl terminus of a TEM. Other non-limiting examples include, locating a targeting domain between a Clostridial toxin enzymatic domain and a Clostridial toxin translocation domain of a TEM. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a targeting domain is in the amino-terminal position, an amino acid sequence comprising a start methionine and a protease cleavage site may be operably-linked in situations in which a targeting domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

A TEM disclosed herein may optionally comprise an exogenous protease cleavage site that allows the use of an exogenous protease to convert the single-chain polypeptide form of a TEM into its more active di-chain form. As used herein, the term “exogenous protease cleavage site” is synonymous with a “non-naturally occurring protease cleavage site” or “non-native protease cleavage site” and means a protease cleavage site that is not naturally found in a di-chain loop region from a naturally occurring Clostridial toxin.

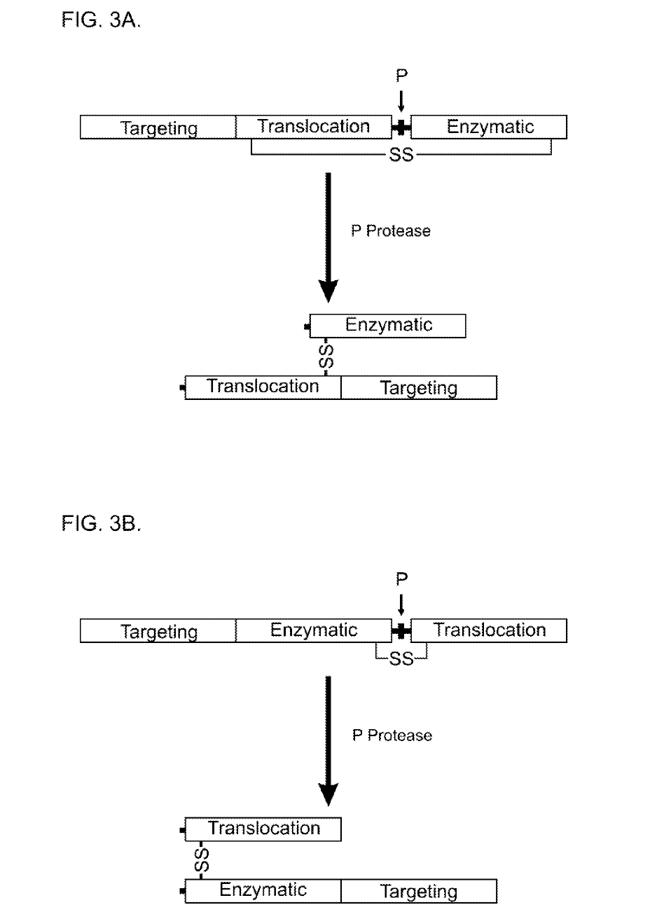

Naturally-occurring Clostridial toxins are each translated as a single-chain polypeptide of approximately 150 kDa that is subsequently cleaved by proteolytic scission within a disulfide loop by a naturally-occurring protease (FIG. 2). This cleavage occurs within the discrete di-chain loop region located between two cysteine residues that form a disulfide bridge and comprising an endogenous protease cleavage site. As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin. This posttranslational processing yields a di-chain molecule comprising an approximately 50 kDa light chain, comprising the enzymatic domain, and an approximately 100 kDa heavy chain, comprising the translocation and cell binding domains, the light chain and heavy chain being held together by the single disulfide bond and non-covalent interactions (FIG. 2). Recombinantly-produced Clostridial toxins generally substitute the naturally-occurring di-chain loop protease cleavage site with an exogenous protease cleavage site to facilitate production of a recombinant di-chain molecule (FIGS. 3-5). See e.g., Dolly, J. O. et al., Activatable Clostridial Toxins, U.S. Pat. No. 7,419,676 (Sep. 2, 2008), which is hereby incorporated by reference.

Although TEMs vary in their overall molecular weight because the size of the targeting domain, the activation process and its reliance on an exogenous cleavage site is essentially the same as that for recombinantly-produced Clostridial toxins. See e.g., Steward, et al., Activatable Clostridial Toxins, US 2009/0081730; Steward, et al., Modified Clostridial Toxins with Enhanced Translocation Capabilities and Altered Targeting Activity For Non-Clostridial Toxin Target Cells, U.S. patent application Ser. No. 11/776,075; Steward, et al., Modified Clostridial Toxins with Enhanced Translocation Capabilities and Altered Targeting Activity for Clostridial Toxin Target Cells, US 2008/0241881, each of which is hereby incorporated by reference. In general, the activation process that converts the single-chain polypeptide into its di-chain form using exogenous proteases can be used to process TEMs having a targeting domain organized in an amino presentation, central presentation, or carboxyl presentation arrangement. This is because for most targeting domains the amino-terminus of the moiety does not participate in receptor binding. As such, a wide range of protease cleavage sites can be used to produce an active di-chain form of a TEM. However, targeting domains requiring a free amino-terminus for receptor binding require a protease cleavage site whose scissile bond is located at the carboxyl terminus. The use of protease cleavage site is the design of a TEM are described in, e.g., Steward, et al., Activatable Clostridial toxins, US 2009/0069238; Ghanshani, et al., Modified Clostridial Toxins Comprising an Integrated Protease Cleavage Site-Binding Domain, US 2011/0189162; and Ghanshani, et al., Methods of Intracellular Conversion of Single-Chain Proteins into their Di-chain Form, International Patent Application Serial No. PCT/US2011/22272, each of which is incorporated by reference in its entirety.

Non-limiting examples of exogenous protease cleavage sites include, e.g., a plant papain cleavage site, an insect papain cleavage site, a crustacian papain cleavage site, an enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a Caspase 3 cleavage site.

Thus, in an embodiment, a TEM can comprise an amino to carboxyl single polypeptide linear order comprising a targeting domain, a translocation domain, an exogenous protease cleavage site and an enzymatic domain (FIG. 3A). In an aspect of this embodiment, a TEM can comprise an amino to carboxyl single polypeptide linear order comprising a targeting domain, a Clostridial toxin translocation domain, an exogenous protease cleavage site and a Clostridial toxin enzymatic domain.

In another embodiment, a TEM can comprise an amino to carboxyl single polypeptide linear order comprising a targeting domain, an enzymatic domain, an exogenous protease cleavage site, and a translocation domain (FIG. 3B). In an aspect of this embodiment, a TEM can comprise an amino to carboxyl single polypeptide linear order comprising a targeting domain, a Clostridial toxin enzymatic domain, an exogenous protease cleavage site, a Clostridial toxin translocation domain.

In yet another embodiment, a TEM can comprise an amino to carboxyl single polypeptide linear order comprising an enzymatic domain, an exogenous protease cleavage site, a targeting domain, and a translocation domain (FIG. 4A). In an aspect of this embodiment, a TEM can comprise an amino to carboxyl single polypeptide linear order comprising a Clostridial toxin enzymatic domain, an exogenous protease cleavage site, a targeting domain, and a Clostridial toxin translocation domain.

In yet another embodiment, a TEM can comprise an amino to carboxyl single polypeptide linear order comprising a translocation domain, an exogenous protease cleavage site, a targeting domain, and an enzymatic domain (FIG. 4B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

In another embodiment, a TEM can comprise an amino to carboxyl single polypeptide linear order comprising an enzymatic domain, a targeting domain, an exogenous protease cleavage site, and a translocation domain (FIG. 4C). In an aspect of this embodiment, a TEM can comprise an amino to carboxyl single polypeptide linear order comprising a Clostridial toxin enzymatic domain, a targeting domain, an exogenous protease cleavage site, a Clostridial toxin translocation domain.

In yet another embodiment, a TEM can comprise an amino to carboxyl single polypeptide linear order comprising a translocation domain, a targeting domain, an exogenous protease cleavage site and an enzymatic domain (FIG. 4D). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

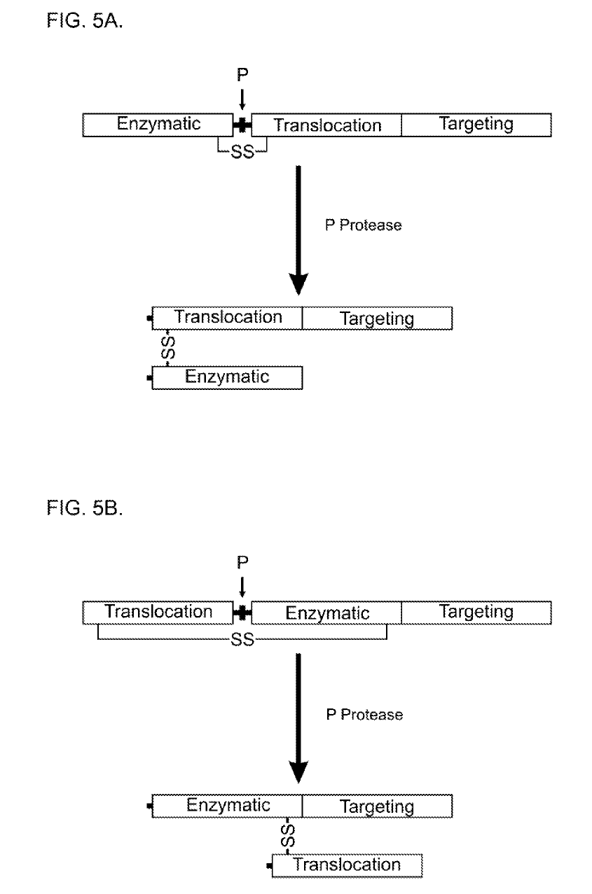

In still another embodiment, a TEM can comprise an amino to carboxyl single polypeptide linear order comprising an enzymatic domain, an exogenous protease cleavage site, a translocation domain, and a targeting domain (FIG. 5A). In an aspect of this embodiment, a TEM can comprise an amino to carboxyl single polypeptide linear order comprising a Clostridial toxin enzymatic domain, an exogenous protease cleavage site, a Clostridial toxin translocation domain, and a targeting domain.

In still another embodiment, a TEM can comprise an amino to carboxyl single polypeptide linear order comprising a translocation domain, an exogenous protease cleavage site, an enzymatic domain and a targeting domain, (FIG. 5B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

Non-limiting examples of TEMs disclosed herein, including TEMs comprising a Clostridial toxin enzymatic domain, a Clostridial toxin translocation domain and a targeting domain, the use of an exogenous protease cleavage site, and the design of amino presentation, central presentation and carboxyl presentation TEMs are described in, e.g., U.S. Pat. No. 7,959,933, Activatable Recombinant Neurotoxins, U.S. Pat. No. 7,897,157, Activatable Clostridial Toxins; U.S. Pat. No. 7,833,535, Clostridial Toxin Derivatives and Methods for Treating Pain; U.S. Pat. No. 7,811,584, Multivalent Clostridial Toxins; U.S. Pat. No. 7,780,968, Clostridial Toxin Derivatives and Methods for Treating Pain; U.S. Pat. No. 7,749,514, Activatable Clostridial Toxins, U.S. Pat. No. 7,740,868, Activatable Clostridial Toxins; U.S. Pat. No. 7,736,659, Clostridial Toxin Derivatives and Methods for Treating Pain; U.S. Pat. No. 7,709,228, Activatable Recombinant Neurotoxins; U.S. Pat. No. 7,704,512, Clostridial Toxin Derivatives and Methods for Treating Pain; U.S. Pat. No. 7,659,092, Fusion Proteins; U.S. Pat. No. 7,658,933, Non-Cytotoxic Protein Conjugates; U.S. Pat. No. 7,622,127, Clostridial Toxin Derivatives and Methods for Treating Pain; U.S. Pat. No. 7,514,088, Multivalent Clostridial Toxin Derivatives and Methods of Their Use; U.S. Pat. No. 7,425,338, Clostridial Toxin Derivatives and Methods for Treating Pain; U.S. Pat. No. 7,422,877, Activatable Recombinant Neurotoxins; U.S. Pat. No. 7,419,676, Activatable Recombinant Neurotoxins; U.S. Pat. No. 7,413,742, Clostridial Toxin Derivatives and Methods for Treating Pain; U.S. Pat. No. 7,262,291, Clostridial Toxin Derivatives and Methods for Treating Pain; U.S. Pat. No. 7,244,437, Clostridial Toxin Derivatives and Methods for Treating Pain; U.S. Pat. No. 7,244,436, Clostridial Toxin Derivatives and Methods for Treating Pain; U.S. Pat. No. 7,138,127, Clostridial Toxin Derivatives and Methods for Treating Pain; U.S. Pat. No. 7,132,259, Activatable Recombinant Neurotoxins; U.S. Pat. No. 7,056,729, Botulinum Neurotoxin-Substance P Conjugate or Fusion Protein for Treating Pain; U.S. Pat. No. 6,641,820, Clostridial Toxin Derivatives and Methods to Treat Pain; U.S. Pat. No. 6,500,436, Clostridial Toxin Derivatives and Methods for Treating Pain; US 2011/0091437, Fusion Proteins; US 2011/0070621, Multivalent Clostridial Toxins; US 2011/0027256, Fusion Proteins; US 2010/0247509, Fusion Proteins; US 2010/0041098, Modified Clostridial Toxins with Altered Targeting Capabilities for Clostridial Toxin Target Cells; US 2010/0034802, Treatment of Pain; US 2009/0162341, Non-Cytotoxic Protein Conjugates; US 2009/0087458, Activatable Recombinant Neurotoxins; US 2009/0081730, Activatable Recombinant Neurotoxins; US 2009/0069238, Activatable Clostridial Toxins; US 2009/0042270, Activatable Recombinant Neurotoxins; US 2009/0030182, Activatable Recombinant Neurotoxins; US 2009/0018081, Activatable Clostridial Toxins; US 2009/0005313, Activatable Clostridial Toxins; US 2009/0004224, Activatable Clostridial Toxins; US 2008/0317783, Clostridial Toxin Derivatives and Methods for Treating Pain; US 2008/0241881, Modified Clostridial Toxins with Enhanced Translocation Capabilities and Altered Targeting Activity for Clostridial Toxin Target Cells; WO 2006/099590, Modified Clostridial Toxins with Altered Targeting Capabilities for Clostridial Toxin Target Cells; WO 2006/101809, Modified Clostridial Toxins with Enhanced Targeting Capabilities for Endogenous Clostridial Toxin Receptor Systems; WO 2007/106115, Modified Clostridial Toxins with Altered Targeting Capabilities for Clostridial Toxin Target Cells; WO 2008/008803, Modified Clostridial Toxins with Enhanced Translocation Capabilities and Altered Targeting Activity for Clostridial Toxin Target Cells; WO 2008/008805, Modified Clostridial Toxins with Enhanced Translocation Capabilities and Altered Targeting Activity For Non-Clostridial Toxin Target Cells; WO 2008/105901, Modified Clostridial Toxins with Enhanced Translocation Capability and Enhanced Targeting Activity; WO 2011/020052, Methods of Treating Cancer Using Opioid Retargeted Endopeptidases; WO 2011/020056, Methods of Treating Cancer Using Galanin Retargeted Endopeptidases; WO 2011/020114, Methods of Treating Cancer Using Tachykinin Retargeted Endopeptidases; WO 2011/020115, Methods of Treating Cancer Using Growth Factor Retargeted Endopeptidases; WO 2011/020117, Methods of Treating Cancer Using Neurotrophin Retargeted Endopeptidases; WO 2011/020119, Methods of Treating Cancer Using Glucagon-Like Hormone Retargeted Endopeptidases; each of which is incorporated entirely by reference.

Aspects of the present specification disclose, in part, a composition. In one aspect of this embodiment, a composition comprises a TEM as disclosed herein. In another aspect of this embodiment, a composition comprises a Clostridial toxin and a TEM as disclosed herein. Any of the compositions disclosed herein can be useful in a method of treating disclosed herein, with the proviso that the composition prevents or reduces a symptom associated with condition being treated. A Clostridial toxin and a TEM as disclosed herein may be provided as separate compositions or as part of a single composition. It is also understood that the two or more different Clostridial toxins and/or TEMs can be provided as separate compositions or as part of a single composition.

A composition disclosed herein is generally administered as a pharmaceutical acceptable composition. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the Clostridial toxins and/or TEMs disclosed herein. A pharmaceutical composition disclosed herein is useful for medical and veterinary applications. A pharmaceutical composition may be administered to an individual alone, or in combination with other supplementary active ingredients, agents, drugs or hormones. The pharmaceutical compositions may be manufactured using any of a variety of processes, including, without limitation, conventional mixing, dissolving, granulating, dragée-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

A pharmaceutical composition disclosed herein may optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active ingredient, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition. Exemplary pharmaceutical composition comprising a TEM are described in Hunt, et al., Animal Protein-Free Pharmaceutical Compositions, U.S. Ser. No. 12/331,816; and Dasari, et al., Clostridial Toxin Pharmaceutical Compositions, WO/2010/090677, each of which is hereby incorporated by reference in its entirety.

In an embodiment, a composition is a pharmaceutical composition comprising a TEM. In aspects of this embodiment, a pharmaceutical composition comprising a TEM further comprises a pharmacological carrier, a pharmaceutical component, or both a pharmacological carrier and a pharmaceutical component. In other aspects of this embodiment, a pharmaceutical composition comprising a TEM further comprises at least one pharmacological carrier, at least one pharmaceutical component, or at least one pharmacological carrier and at least one pharmaceutical component.

In another embodiment, a composition is a pharmaceutical composition comprising a Clostridial toxin. In aspects of this embodiment, a pharmaceutical composition comprising a Clostridial toxin further comprises a pharmacological carrier, a pharmaceutical component, or both a pharmacological carrier and a pharmaceutical component. In other aspects of this embodiment, a pharmaceutical composition comprising a Clostridial toxin further comprises at least one pharmacological carrier, at least one pharmaceutical component, or at least one pharmacological carrier and at least one pharmaceutical component.

In yet another embodiment, a composition is a pharmaceutical composition comprising a Clostridial toxin and a TEM. In aspects of this embodiment, a pharmaceutical composition comprising a Clostridial toxin and a TEM further comprises a pharmacological carrier, a pharmaceutical component, or both a pharmacological carrier and a pharmaceutical component. In other aspects of this embodiment, a pharmaceutical composition comprising a Clostridial toxin and a TEM further comprises at least one pharmacological carrier, at least one pharmaceutical component, or at least one pharmacological carrier and at least one pharmaceutical component.

Aspects of the present specification disclose, in part, treating an individual suffering from a sensory disturbance disorder. As used herein, the term “treating,” refers to reducing or eliminating in an individual a clinical symptom of a sensory disturbance disorder; or delaying or preventing in an individual the onset of a clinical symptom of a sensory disturbance disorder. For example, the term “treating” can mean reducing a symptom of a condition characterized by a sensory disturbance disorder by, e.g., at least 20%, at least 30%, at least 40%, at least 50%, at least 60%, at least 70%, at least 80%, at least 90% or at least 100%. The actual symptoms associated with a sensory disturbance disorder are well known and can be determined by a person of ordinary skill in the art by taking into account factors, including, without limitation, the location of the sensory disturbance disorder, the cause of the sensory disturbance disorder, the severity of the sensory disturbance disorder, and/or the tissue or organ affected by the sensory disturbance disorder. Those of skill in the art will know the appropriate symptoms or indicators associated with specific sensory disturbance disorder and will know how to determine if an individual is a candidate for treatment as disclosed herein.

As used herein, the term “sensory disturbance disorder” refers to a sensory disturbance disorder where at least one of the underlying symptoms being treated is due to a sensory nerve-based etiology, a sympathetic nerve-based etiology, and/or a parasympathetic nerve-based etiology. Typically such etiologies will involve an abnormal overactivity of a nerve that results in symptoms of a sensory disturbance disorder, or any normal activity of a nerve that needs to be reduced or stopped for a period of time in order to treat a sensory disturbance disorder. Sensory disturbance disorders include, without limitation, a sensory processing disorder, an auditory disturbance, an olfactory disturbance, a gustatory disturbance, a visual disturbance, a hallucination disorder, a nausea, a vomiting, and a gastrointestinal dysfunction.

A sensory processing disorder (SPD, or sensory integration disorder, sensory integration dysfunction, or sensory processing dysfunction) refers to a sensory disturbance disorder where an individual has difficulties with or abnormalities in taking in, processing and responding to sensory stimuli, including information about the environment or even the individual's own body. An individual with an SPD may have difficulties with stimuli from one sense or multiple senses, including: auditory (sound), gustatory (taste), olfactory (smell), proprioreceptive (body position in space), tactile (touch), and vestibular (equilibrium and balance) sense. Some individuals are so profoundly affected by SPD they have trouble with daily functioning.

An SPD can be further categorized into subtypes based on diagnostic criteria. These types of SPD include Type I, a sensory modulation disorder; Type II, a sensory-based motor disorder; and, Type III, a sensory discrimination disorder. A Type I SPD, or sensory modulation disorder, refers to a type of SPD where an individual over- or under-responds to one or more sensory stimuli, or experiences a combination of any of these. This can result in the affected individual exhibiting anxious, fearful, self-absorbed, and/or stubborn behaviors, and possibly seeking out particular stimuli. A Type II SPD, or sensory-based motor disorder, refers to a type of SPD where an individual has disorganized motor output due to the incorrect processing of sensory stimuli. This can result in motor planning dyspraxia (i.e., the inability to plan and perform coordinated movements) and problems with the individual's posture control. A Type III SPD, or sensory discrimination disorder, refers to a type of SPD where an individual incorrectly processes sensory stimuli. This can result in the individual appearing to be disorganized and inattentive, and poor performance academically.

Some forms of an SPD are a hypersenstivity or hyposensitivity. A hypersenstivity refers to an SPD where an individual has discomfort with or even feels pain from normal sense stimuli; for example, the touch of clothing or normal lighting. A hyposensitivity refers to an SPD where an individual has an unusually high tolerance for sense stimuli. A child with a hyposensitivity may seek out rough play and appear restless.

An SPD can frequently be a comorbidity with autism spectrum disorders, and can also be a comorbidity with attention-deficit hyperactivity disorder (ADHD). Alternatively, an autism spectrum disorder or ADHD may be misdiagnosed when in fact an individual suffers from an SPD.

An auditory disturbance refers to a sensory disturbance disorder where an individual has a disordered response to auditory stimuli, or perceives auditory stimuli in an abnormal way or perceives auditory stimuli that do not actually exist. Auditory disturbances include, without limitation, a phonophobia and an auditory distortion.

A phonophobia (or ligyrophobia) refers to an auditory disturbance where an individual has a fear, aversion, or is hypersensitive to loud sounds, which could include the sound of voices, including one's own. It is estimated that 15% of the world's population has some form of phonophobia.

An auditory distortion (or auditory dysfunction) is a generic term that refers to a variety of possible distortions in an individual's perception of auditory stimuli. Auditory distortions include, without limitation, hypersensitivity to sound (auditory defensiveness), hyposensitivity to sound (under-registers),

An olfactory disturbance refers to a sensory disturbance disorder where an individual has a disordered response to olfactory stimuli or perceives olfactory stimuli in an abnormal way or perceives olfactory stimuli that do not actually exist. Olfactory disturbances include, without limitation, an osmophobia and an olfactory distortion.

Osmophobia (or olfactophobia) refers to an olfactory disturbance where an individual has a fear of or aversion to smells, or is hypersensitive to smells. The phobia generally occurs in chronic migraine sufferers, whose migraines may be triggered by certain odors. The triggering odors are most frequently foul odors, but the osmophobic hypersensitivity may extend to all odors. A correlation has also been shown between migraine sufferers in general and osmophobia. One possible treatment for an osmophobia is administering pleasant odors, such as lavender or mint.

An olfactory distortion (or olfactory dysfunction, or dysosmia) refers to an olfactory disturbance where an individual has an impairment in the processing of olfactory stimuli, which leads to an altered or disordered sense of smell. Some examples of an olfactory distortion include, without limitation: an anosmia, a hyposmia, a hyperosmia, and a parosmia.

An anosmia refers to an olfactory distortion where an individual is unable to perceive odors. An anosmia can relate to the inability to perceive a specific odor or several odors. There can be a variety of causes for an anosmia, including a physical obstruction in the nasal passageways, infection, inflammation, neurological damage, genetics and congenital defects. An anosmia can be acute or chronic. An acute anosmia is generally associated with an obstruction or infection. A chronic anosmia is generally associated with neurological damage. Some individuals (anosmics) are born with an anosmia.

A hyposmia refers to an olfactory distortion where an individual has a decreased ability to smell. A hyperosmia refers to an olfactory distortion where an individual has an increased ability to smell odors.

A parosmia (or cacosmia or troposmia) is an olfactory distortion where an individual incorrectly identifies odors. An individual with a parosmia may describe a pleasant odor (for example, mint) as smelling burned, fecal, rotten, etc. A parosmia may relate to the misinterpretation of a specific odor, or several odors.

A gustatory disturbance (a distortion in the sense of taste), in which an individual has an altered or disordered sense of taste. In some cases a gustatory disturbance can be considered a subset of an olfactory disturbance, similar to an olfactory distortion, because the sense of taste in general is mostly derived from the sense of smell. Some examples of a gustatory disturbance include, without limitation: an ageusia, a hypogeusia, a hypergeusia, and a dysgeusia.

An ageusia refers to a gustatory distortion where an individual has a complete loss of taste. A hypogeusia refers to a gustatory distortion where an individual has a partial loss of taste. A dysgeusia refers to a gustatory distortion where an individual perceives a persistent abnormal taste in the mouth, or a distortion of taste (a parageusia being the medical term for having a bad taste in the mouth, such as a metallic taste).

A hypogeusia refers to a gustatory distortion where an individual has a partial loss of taste. A hypergeusia refers to a gustatory distortion where an individual has an abnormally heightened sense of taste. A hypergeusia can be a symptom of, for example, a lesion in the posterior fossa, or Addison's Disease.

A visual disturbance refers to a sensory disturbance disorder where an individual has a disordered response to visual stimuli or perceives visual stimuli in an abnormal way or perceives visual stimuli that do not actually exist. Visual disturbances include, without limitation, a photophobia and a visual distortion.

A photophobia (or light sensitivity) refers to a visual disturbance where an individual has a fear of or aversion to light, or is hypersensitive to light. A photohobia can be a response to a bright light or in some cases any light. The light which causes the photophobia can be from any light source, including fluorescent or incandescent light or sunlight. Besides the light response, the photophobia sufferer can also experience discharge from the eyes; dizziness; headaches or shooting pains in the temples; nausea; pain in the eyes; redness in the eyes; the need to squint or close the eyes; swelling of the eyes; and/or, a stiff neck. A photophobia can arise, for example but without limitation, from the optic nerve receiving excessive electric impulses, an excessive response within the central nervous system, and/or overstimulation of retinal photoreceptors.

A photophobia is generally not considered an eye disease per se, but a symptom of some other condition, disorder, disease, trauma, etc. Accordingly, the potential causes of a photophobia are numerous and varied, and can include, for example but without limitation: aniridia; use of anticholinergic drugs, which may paralyze the iris sphincter muscle and thus prohibit normal pupil constriction; aphakia (absence of the lens of the eye); ariboflavinosis; an Arnold-Chiari (or simply Chiari) malformation of the brain; atrophy of the optic nerve, for example as can be caused by excessive use of alcohol; autism spectrum disorders; botulism; buphthalmos (abnormally narrow angle between the iris and cornea); cataracts; a disorder of the central nervous system, such as for example meningitis; a chalazion; chemotherapy; Chikungunya virus (CHIKV); some forms of color blindness, especially total color deficiency wherein the individual only sees black and white and shades of gray (achromatopsia); cone dystrophy; congenital eye defects or genetic disorders, such as keratosis follicularis spinulosa decalvans; congenital glaucoma (hydrophthalmos); conjunctivitis, such as viral conjunctivitis (pink eye); irritations from contact lenses; corneal abrasions or ulcers; corneal dystrophy; disruption of the corneal epithelium, for example, such as due to keratitis or a foreign body; cystinosis; dyslexia; ectopia lentis; Ehlers-Danlos syndrome; encephalitis, including myalgic encephalomyelitis (or chronic fatigue syndrome); endophthalmitis; episcleritis; eye trauma; glaucoma; hangover; influenza; any of a variety of infections; infectious mononucleosis; inflammation, such as irritation of the brain and/or other nerves, for example as can be caused by excessive use of alcohol; iritis; keratitis; keratoconus; having lighter colored eyes or albinism, due to the irises containing less or no pigment to protect against the light; magnesium deficiency; use of or withdrawal from some medications, such as for example doxycycline, quinine, and tetracycline; mercury poisoning; migraines or other severe headaches; optic nerve hypoplasia; optic neuritis; pigment dispersion syndrome; pupil dilation, natural or chemically induces, or the inability of the pupil to constrict normally, for example as can be caused by damage to the oculomotor nerve, paralysis of the iris sphincter muscle, etc.; retinal detachment; rabies; refractive surgery; detached retina; scarring of the cornea or sclera; subarachnoid hemorrhage; sunburn; tumor of the posterior cranial fossa; Type II tyrosinemia (or Richner-Hanhart syndrome); and, uveitis, for example as due to ankylosing spondylitis.

A visual distortion refers to a visual disorder where an individual has an impairment in the ability to receive and process visual stimuli. A visual distortion can be temporary, recurrent, chronic or permanent. There are a variety of types of vision distortion. Some examples include, without limitation: blind spots; blindness; cloudy vision; a distortion of shapes; seeing double vision; seeing flashes; seeing floaters; seeing halos; an impairment in depth perception; an impairment in peripheral vision; and, an impairment in night vision.

A visual distortion can be caused by a variety of conditions, diseases or disorders, which can originate in the eyes or elsewhere in the body. Some examples of causes of a visual distortion include, without limitation: brain hemorrhage, botulism, cancer or tumor, carotid embolism, cataracts, corneal ulcer, dry eyes, encephalitis, epilepsy, glaucoma, headache or migraine, infection (e.g. conjunctivitis), inflammation, injury or trauma, iritis, irritation of the eyes, macular degeneration, medications, multiple sclerosis, myasthenia gravis, optic neuritis, orbital cellulitis, poisoning, pregnancy, psychosis, refractive errors (farsightedness or nearsightedness) due to an irregularly shaped cornea, retinal detachment, sarcoidosis, stroke, systemic lupus erythematosis, temporal arteritis, transient ischemic attack, and uveitis.

A visual distortion can also be accompanied by other symptoms, which could indicate a serious underlying medical condition. Some of these associated symptoms can include confusion, discharge from the eyes, eyelid swelling, fainting, hallucinations, headache, inability to move some part of the body, red eyes, slurred speech, and stiff neck. Complications that could be associated with a visual distortion, especially if the underlying cause of the visual distortion is left untreated, can include for example wasting and/or permanent deformities of the arms or legs, permanent brain and/or nervous system damage, blindness, coma, loss of the eye, and seizures.

A hallucination refers to a sensory disturbance disorder where an individual experiences sensory perceptions in the absence of an actual, external stimuli. The perceptions are real to the individual, in the sense that they occur while the individual is awake (not dreaming), and are vivid and located in objective space. The function (or dysfunction) of the neurotransmitters glutamate and dopamine are thought to be particularly important in contributing to the manifestation of a hallucination. A hallucination can occur from perception of any of the senses: auditory, chronoceptive (perception of time), equilibrioceptive (sense of balance), gustatory, nociceptive (perception of pain or other noxious stimuli), olfactory, proprioceptive (sense of the position of one's body), tactile, thermoceptive, and/or visual.

An auditory hallucination (or paracusia) refers to a hallucination wherein an individual perceives sound in the absence of external auditory stimulus. Auditory hallucinations can be further categorized as elementary or complex. An elementary auditory hallucination refers to the perception of a simple sound such as a particular tone, a hissing, etc. In some but not all cases tinnitus is an elementary auditory hallucination. A complex auditory hallucination refers to the perception of complex sounds such as music, talking voices, or other unclear sounds. Examples of possible causes of an auditory hallucination include, without limitation: hearing loss (Musical Ear Syndrome), schizophrenia or other psychiatric illnesses, lateral temporal lobe epilepsy or other epilepsy, arteriovenous malformation, stroke, lesion, abscess, tumor, listening to music for long periods of time, lesions on the brain stem, encephalitis, and mood disorders. Where an auditory hallucination is related to schizophrenia, individuals tend to demonstrate a consistent increase in activity of the hypothalamus, paralimbic regions, and thalamic and strietal subcortical nuclei, which would imply that functional and/or structural brain abnormalities can induce an auditory hallucination, and that there could be a genetic component.

A gustatory hallucination refers to a hallucination where an individual perceives taste in the absence of gustatory (or olfactory) stimuli. The perceived taste is usually strange or unpleasant. A gustatory hallucination can be associated with certain types of focal epilepsy, such as temporal lobe epilepsy, in which case the responsible areas of the brain are the insula and the superior bank of the sylvian fissure.

An olfactory hallucination (phantosmia) refers to a hallucination where an individual perceives smells in the absence of olfactory stimuli. Most commonly, perceived smells are unpleasant, such as feces, rotting flesh, smoke, vomit, urine, etc. An olfactory hallucination can be caused by damage to the nervous tissue in the olfactory system from any of a variety of causes, including without limitation: medications, poisoning, surgery, tumor, and viral infection. An olfactory hallucination can also be caused by epilepsy, which affects the olfactory cortex, or migraines.

A tactile hallucination refers to a hallucination where an individual perceives tactile stimuli in the absence of real stimuli; for example, pressure on the skin or other organs. One particular example of a tactile hallucination is a formication, in which an individual perceives insects crawling on or underneath the skin. Some examples of causes of a formication include, without limitation: cocaine or amphetamine use, withdrawal from alcohol or benzodiazepine use, hormonal changes such as menopause, peripheral neuropathy or like disorders, high fever, Lyme disease, and skin cancer.

A visual hallucination refers to a hallucination where an individual perceives visions in the absence of visual stimuli. Causes of a visual hallucination can be categorized as psychobiochemical, which relates to a disturbance of the neurotransmitters; psychological; and, psychophysiologic, which relates to a disturbance of brain structure. Examples of possible causes of a visual hallucination include, without limitation: alcohol use, any of various organic disorders of the brain, dementia, drug use, migraine, psychotic disorders.

Some examples of specific types of hallucinations and their causes include, without limitation: a hypnagogic hallucination (hypnagogia), which generally occurs when an individual is drowsy and just before falling asleep, and can be associated with narcolepsy or brainstem abnormalities; a peduncular hallucinosis, which is a hallucination pertaining to the peduncle (a neural tract running from the pons on the brain stem), usually occurring at night but not during drowsiness; a hallucination of delirium tremens; a hallucination of Parkinson's disease or of Lewy body dementia, in the case of Parkinson's disease usually associated with a degradation of the substantia nigra pars compacta, median raphe nuclei, noradrenergic parts of the locus coeruleus, and the cholinergic neurons in the parabrachial and pedunculopontine nuclei of the tegmentum; a migraine coma hallucination, in which the hallucination is experienced while recovering from a coma, often with ataxic lesions accompany the coma; a Charles Bonnet syndrome hallucination, which is a visual hallucination experienced by the blind; a focal epilepsy hallucination, in which the particular visions perceived will differ based on the region of the brain where the seizure occurs.

A nausea refers to a sensory disturbance disorder where an individual has a sensation of discomfort, queasiness or unease in the upper stomach, with the feeling of an urge to vomit. These sensations can vary in intensity; e.g., from mild to moderate to extreme intensity. Nausea may or may not be accompanied by vomiting. Nausea is also generally accompanied by a distaste for food and/or inability to eat, and may be accompanied by other symptoms such as salivation, tachycardia, pallor, tachypnea, diaphoresis and pallor. Nauseas include, without limitation: an acute nausea, a short-term nausea, a persistent nausea, a chronic nausea, a recurring nausea, an unexplained nausea, a breakthrough nausea, and a refractory nausea.

An acute nausea refers to nausea which begins suddenly, quickly worsens, and then lasts for a relatively brief period. A short-term nausea refers to a nausea which is of relatively short duration and may even be a fleeting sensation. A persistent (or constant) nausea refers to a nausea which manifests and then does not cease for a period of time. A recurring nausea is a nausea in which the symptoms appear, cease, and then reappear repeatedly for a period of time. A chronic (or ongoing) nausea refers to a nausea which continues over an extended period of time, perhaps indefinitely. An individual with chronic nausea may suffer the symptoms of nausea constantly (persistent nausea), or the symptoms may be intermittent (recurring nausea); for example, the nausea may persist for several weeks, or may recur daily. An unexplained nausea is a nausea in which the individual who suffers the nausea is unaware of the cause or unable to discern the cause of the nausea. A breakthrough nausea refers to a nausea which occurs despite the fact that an individual is specifically treated for the prevention of nausea. A refractory nausea refers to a nausea which is resistant to ordinary methods of treatment, or which no longer responds to treatment.

A vomiting (or emesis) refers to a sensory disturbance disorder where an individual forcefully disgorges all or some of the stomach contents, and possibly intestinal contents (collectively, vomitus) through the mouth and/or possibly the nose. Alternatively, a vomiting as used herein refers to a sensory disturbance disorder where an individual experiences a retching but without actual production of vomitus (also known as dry heaves). A vomiting may be but is not always preceded or accompanied by nausea. A vomiting can include, without limitation: an acute vomiting, a short-term vomiting, a persistent vomiting, a chronic vomiting, a recurring vomiting, an unexplained vomiting, a breakthrough vomiting, a refractory vomiting, and a retching.

An acute vomiting refers to a vomiting episode which begins suddenly, quickly worsens, and then lasts for a relatively brief period. A short-term vomiting refers to an episode of vomiting which is of relatively short duration. A persistent (or constant) vomiting refers to a vomiting in which an individual begins vomiting and then is unable to stop for a period of time. A recurring vomiting (or cyclic vomiting) is a vomiting in which an individual has a vomiting episode, the vomiting ceases, and then recurs repeatedly for a period of time. A chronic (or ongoing) vomiting refers to a vomiting in which the individual experiences episodes that continue over an extended period of time, perhaps indefinitely. An individual with chronic vomiting may suffer vomiting constantly (persistent vomiting), or the vomiting may be intermittent (recurring vomiting); for example, the vomiting may persist for several weeks, or may recur daily. An unexplained vomiting is a vomiting in which the individual who suffers the vomiting is unaware of the cause or unable to discern the cause of the vomiting. A breakthrough vomiting refers to a vomiting which occurs despite the fact that an individual is specifically treated for the prevention of vomiting. A refractory vomiting refers to a vomiting which is resistant to ordinary methods of treatment, or which no longer responds to treatment.

A vomiting that includes blood is termed hematemesis. A vomiting that includes matter from the intestines digested by the stomach is termed fecal vomiting (or stercoraceous vomiting or copremesis). Fecal vomiting usually leads to severe aspiration pneumonia, and can be fatal. Projectile vomiting refers to a vomiting where vomitus is ejected with great force.

There are several harmful side effects that can be associated with vomiting, especially in recurring, repeated or profuse vomiting. These include tears in the esophageal mucosa (termed a Mallory-Weiss tear) and/or erosions to the esophagus. Both of these, if sufficiently extensive, can result in blood in the vomitus. Destruction of the tooth enamel and a degradation of the gum tissue can also result due to the digestive enzymes and acids from the stomach that are present in the vomitus. The individual may aspirate the vomitus, which can result in pneumonia or even asphyxiation. The individual can become dehydrated and/or suffer an electrolyte imbalance. With continued vomiting, the individual can eventually become cachectic due to loss of nourishment.

Cyclic vomiting syndrome (CVS, or cyclical vomiting syndrome) is a disorder characterized by recurring episodes (at least three or more) of intense nausea and vomiting, accompanied by headaches and abdominal pain, with intervening periods without symptoms lasting weeks or months. During each vomiting episode the CVS sufferer may vomit six to twelve times an hour. These vomiting episodes can last from a few hours to several weeks, and in some cases even months. Prior to each episode, some sufferers experience a prodrome that usually includes intense nausea and pallor. Prior to each episode individuals may also be sensitive to light (photophobic), pressure, smell, sound (phonophobic), and/or temperature, and experience fatigue and muscle pain. During a vomiting attack, acid, bile and, in severe cases blood may be vomited. The individual may also be sensitive to light, pressure, sound, and/or temperature during the attack. Individuals may also experience a restless sensation or pain in the feet, hands, and/or spine, and may be weak in the legs. In cases of very extensive vomiting episodes, fluid loss can be so severe as to lead to life-threatening electrolyte imbalances. Extremely high blood pressure also often develops during an episode. The CVS sufferer may become undernourished if the episode lasts long enough. Between episodes the CVS sufferer may be in a weakened state, be fatigued, and experience muscle pain. In the developed world with adequate medical interventions most sufferers can be supported during an attack and will recover from the episode

CVS generally manifests in early childhood, usually from age three to seven; however, it may also arise in infants or the elderly. In some cases it remits in adolescence, but in others it persists into adulthood. The cause of CVS is unknown, but there appears to be a genetic component. For example, it has been observed that many individuals affected with CVS have a family history in their maternal relatives of related conditions such as migraines. This would suggest mitochondrial inheritance of CVS. Additionally, most CVS sufferers can generally identify triggers that will precipitate an attack, which include certain foods, infections such as colds, menstruation, physical exertion, lack of sleep, and stress.

Studies of CVS suggest nearly 2% of school age children have CVS, but with diagnosis problematic and the recent increased recognition of CVS, more cases are emerging, such that the tendency may be to underdiagnose and the true percentage of individuals suffering from CVS may be higher. There is no known cure for CVS. Treatment is usually based on trial and error, and is limited to maintaining salt and fluid balance during an episode, including intravenously, sedation, painkillers, and powerful anti-emetic drugs. Besides the costs to the individual's health, there are also societal costs associated with CVS, such as missing school or work. In underdeveloped countries and if left untreated, the CVS symptoms described above may contribute to mortality.

A gastrointestinal dysfunction refers to a sensory disturbance disorder where an individual has a dysfunction in the digestive tract. Gastrointestinal dysfunctions include, without limitation, a diarrhea or a constipation.

A diarrhea refers to a gastrointestinal dysfunction where an individual has three or more loose or bowel movements per day. A diarrhea is a common cause of mortality in developing countries and of infant deaths worldwide. The loss of fluids that result from a diarrhea can result in dehydration and electrolyte imbalances. Treatments for the effects of a diarrhea include oral rehydration salts and zinc tablets. Some examples of causes of a diarrhea include, without limitation: alcohol use, bile salt malabsorption, hormone-secreting tumors, infection, inflammatory bowel disease, irritable bowel syndrome, ischemic bowel disease, malabsorption (inability to absorb food fully or maldigestion), microscopic colitis, toddler's diarrhea (diarrhea in infants and toddlers with no known cause).

A constipation refers to a gastrointestinal dysfunction where an individual has infrequent or hard to pass bowel movements. If left untreated, a constipation can lead to the failure to pass stools or gas, and fecal impaction.

Examples of some causes of a constipation include, without limitation: anal fissures, anismus, celiac disease, colon cancer, colorectal cancer, cystic fibrosis, descending perineum syndrome, diabetes mellitus, dieting, insufficient dietary fiber, insufficient fluid, Hirschsprung's disease, hypercalcemia, hypothyroidism, medications (such as antacids, anticonvulsants, antidepressants, antihistamines, antispasmodics, diuretics, and opioids), muscular dystrophy, myotonic dystrophy, Parkinson's disease, decreased physical activity, proctitis, pelvic floor dysfunction, spinal cord lesions. A constipation can also occur as a result of voluntary actions of the affected individual, such as withholding of the stool due to laziness or fear (of pain, of public restrooms, etc.).

A composition or compound is administered to an individual. An individual comprises all mammals including a human being. Typically, any individual who is a candidate for a conventional sensory disturbance disorder treatment is a candidate for a sensory disturbance disorder treatment disclosed herein. Pre-operative evaluation typically includes routine history and physical examination in addition to thorough informed consent disclosing all relevant risks and benefits of the procedure.

With reference to a therapy comprising a TEM, the amount of a TEM disclosed herein used with the methods of treatment disclosed herein will typically be an effective amount. As used herein, the term “effective amount” is synonymous with “therapeutically effective amount”, “effective dose”, or “therapeutically effective dose” and when used in reference to treating a sensory disturbance disorder means the minimum dose of a TEM alone necessary to achieve the desired therapeutic effect and includes a dose sufficient to reduce a symptom associated with a sensory disturbance disorder. An effective amount refers to the total amount of a TEM administered to an individual in one setting. As such, an effective amount of a TEM does not refer to the amount administered per site. The effectiveness of a TEM disclosed herein in treating a sensory disturbance disorder can be determined by observing an improvement in an individual based upon one or more clinical symptoms, and/or physiological indicators associated with the condition. An improvement in a sensory disturbance disorder also can be indicated by a reduced need for a concurrent therapy.

With reference to a standard dose combination therapy comprising a Clostridial toxin and a TEM, an effective amount of a Clostridial toxin is one where in combination with a TEM the amount of a Clostridial toxin achieves the desired therapeutic effect. For example, typically about 75-150 U of BOTOX® (Allergan, Inc., Irvine, Calif.), a BoNT/A, is administered in order to treat a sensory disturbance disorder.

With reference to a low dose combination therapy comprising a Clostridial toxin and a TEM, an effective amount of a Clostridial toxin is one where in combination with a TEM the amount of a Clostridial toxin achieves the desired therapeutic effect, but such an amount administered on its own would be ineffective. For example, typically about 75-150 U of BOTOX® (Allergan, Inc., Irvine, Calif.), a BoNT/A, is administered in order to treat a sensory disturbance disorder. However, in a low dose combination therapy, a suboptimal effective amount of BoNT/A would be administered to treat a sensory disturbance disorder when such toxin is used in a combined therapy with a TEM. For example, less that 50 U, less than 25 U, less than 15 U, less than 10 U, less than 7.5 U, less than 5 U, less than 2.5 U, or less than 1 U of BoNT/A would be administered to treat a sensory disturbance disorder when used in a low dose combination therapy with a TEM as disclosed herein.

The appropriate effective amount of a Clostridial toxin and/or a TEM to be administered to an individual for a particular sensory disturbance disorder can be determined by a person of ordinary skill in the art by taking into account factors, including, without limitation, the type of sensory disturbance disorder, the location of the sensory disturbance disorder, the cause of the sensory disturbance disorder, the severity of the sensory disturbance disorder, the degree of relief desired, the duration of relief desired, the particular TEM and/or Clostridial toxin used, the rate of excretion of the particular TEM and/or Clostridial toxin used, the pharmacodynamics of the particular TEM and/or Clostridial toxin used, the nature of the other compounds to be included in the composition, the particular route of administration, the particular characteristics, history and risk factors of the individual, such as, e.g., age, weight, general health and the like, or any combination thereof. Additionally, where repeated administration of a composition disclosed herein is used, an effective amount of a Clostridial toxin and/or a TEM will further depend upon factors, including, without limitation, the frequency of administration, the half-life of the particular TEM and/or Clostridial toxin used, or any combination thereof. In is known by a person of ordinary skill in the art that an effective amount of a composition comprising a Clostridial toxin and/or TEM can be extrapolated from in vitro assays and in vivo administration studies using animal models prior to administration to humans.

Wide variations in the necessary effective amount are to be expected in view of the differing efficiencies of the various routes of administration. For instance, oral administration generally would be expected to require higher dosage levels than administration by intravenous or intravitreal injection. Similarly, systemic administration of a TEM would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a TEM disclosed herein that is administered can be adjusted accordingly.

In aspects of this embodiment, a therapeutically effective amount of a composition comprising a TEM reduces a symptom associated with a sensory disturbance disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TEM reduces a symptom associated with a sensory disturbance disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TEM reduces a symptom associated with a sensory disturbance disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of the TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a TEM generally is in the range of about 1 fg to about 3.0 mg. In aspects of this embodiment, an effective amount of a TEM can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a TEM can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a TEM can be, e.g., at least 1 fg, at least 250 fg, at least 500 fg, at least 750 fg, at least 1 μg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TEM can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

In yet other aspects of this embodiment, a therapeutically effective amount of a TEM generally is in the range of about 0.00001 mg/kg to about 3.0 mg/kg. In aspects of this embodiment, an effective amount of a TEM can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a TEM can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a TEM can be, e.g., at most 0.00001 mg/kg, at most 0.0001 mg/kg, at most 0.001 mg/kg, at most 0.01 mg/kg, at most 0.1 mg/kg, or at most 1 mg/kg.

In aspects of this embodiment, a therapeutically effective amount of a composition comprising a Clostridial toxin reduces a symptom associated with a sensory disturbance disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a Clostridial toxin reduces a symptom associated with a sensory disturbance disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a Clostridial toxin reduces a symptom associated with a sensory disturbance disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a Clostridial toxin is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a Clostridial toxin generally is in the range of about 1 fg to about 30.0 μg. In other aspects of this embodiment, a therapeutically effective amount of a Clostridial toxin can be, e.g., at least 1.0 pg, at least 10 pg, at least 100 pg, at least 1.0 ng, at least 10 ng, at least 100 ng, at least 1.0 μg, at least 10 μg, at least 100 μg, or at least 1.0 mg. In still other aspects of this embodiment, a therapeutically effective amount of a Clostridial toxin can be, e.g., at most 1.0 pg, at most 10 pg, at most 100 pg, at most 1.0 ng, at most 10 ng, at most 100 ng, at most 1.0 μg, at most 10 μg, at most 100 μg, or at most 1.0 mg. In still other aspects of this embodiment, a therapeutically effective amount of a Clostridial toxin can be, e.g., about 1.0 pg to about 10 μg, about 10 pg to about 10 μg, about 100 pg to about 10 μg, about 1.0 ng to about 10 μg, about 10 ng to about 10 μg, or about 100 ng to about 10 μg. In still other aspects of this embodiment, a therapeutically effective amount of a Clostridial toxin can be from, e.g., about 1.0 pg to about 1.0 μg, about 10 pg to about 1.0 μg, about 100 pg to about 1.0 μg, about 1.0 ng to about 1.0 μg, about 10 ng to about 1.0 μg, or about 100 ng to about 1.0 μg. In other aspects of this embodiment, a therapeutically effective amount of a Clostridial toxin can be from, e.g., about 1.0 pg to about 100 ng, about 10 pg to about 100 ng, about 100 pg to about 100 ng, about 1.0 ng to about 100 ng, or about 10 ng to about 100 ng.

In yet other aspects of this embodiment, a therapeutically effective amount of a Clostridial toxin generally is in the range of about 0.1 U to about 2500 U. In other aspects of this embodiment, a therapeutically effective amount of a Clostridial toxin can be, e.g., at least 1.0 U, at least 10 U, at least 100 U, at least 250 U, at least 500 U, at least 750 U, at least 1,000 U, at least 1,500 U, at least 2,000 U, or at least 2,500 U. In still other aspects of this embodiment, a therapeutically effective amount of a Clostridial toxin can be, e.g., at most 1.0 U, at most 10 U, at most 100 U, at most 250 U, at most 500 U, at most 750 U, at most 1,000 U, at most 1,500 U, at most 2,000 U, or at most 2,500 U. In still other aspects of this embodiment, a therapeutically effective amount of a Clostridial toxin can be, e.g., about 1 U to about 2,000 U, about 10 U to about 2,000 U, about 50 U to about 2,000 U, about 100 U to about 2,000 U, about 500 U to about 2,000 U, about 1,000 U to about 2,000 U, about 1 U to about 1,000 U, about 10 U to about 1,000 U, about 50 U to about 1,000 U, about 100 U to about 1,000 U, about 500 U to about 1,000 U, about 1 U to about 500 U, about 10 U to about 500 U, about 50 U to about 500 U, about 100 U to about 500 U, about 1 U to about 100 U, about 10 U to about 100 U, about 50 U to about 100 U, about 0.1 U to about 1 U, about 0.1 U to about 5 U, about 0.1 U to about 10 U, about 0.1 U to about 15 U, about 0.1 U to about 20 U, about 0.1 U to about 25 U.

In still other aspects of this embodiment, a therapeutically effective amount of a Clostridial toxin generally is in the range of about 0.0001 U/kg to about 3,000 U/kg. In aspects of this embodiment, a therapeutically effective amount of a Clostridial toxin can be, e.g., at least 0.001 U/kg, at least 0.01 U/kg, at least 0.1 U/kg, at least 1.0 U/kg, at least 10 U/kg, at least 100 U/kg, or at least 1000 U/kg. In other aspects of this embodiment, a therapeutically effective amount of a Clostridial toxin can be, e.g., at most 0.001 U/kg, at most 0.01 U/kg, at most 0.1 U/kg, at most 1.0 U/kg, at most 10 U/kg, at most 100 U/kg, or at most 1000 U/kg. In yet other aspects of this embodiment, a therapeutically effective amount of a Clostridial toxin can be between, e.g., about 0.001 U/kg to about 1 U/kg, about 0.01 U/kg to about 1 U/kg, about 0.1 U/kg to about 1 U/kg, about 0.001 U/kg to about 10 U/kg, about 0.01 U/kg to about 10 U/kg, about 0.1 U/kg to about 10 U/kg about 1 U/kg to about 10 U/kg, about 0.001 U/kg to about 100 U/kg, about 0.01 U/kg to about 100 U/kg, about 0.1 U/kg to about 100 U/kg, about 1 U/kg to about 100 U/kg, or about 10 U/kg to about 100 U/kg. As used herein, the term “unit” or “U” is refers to the LD₅₀ dose, which is defined as the amount of a Clostridial toxin disclosed herein that killed 50% of the mice injected with the Clostridial toxin.

In aspects of this embodiment, a therapeutically effective amount of a standard or low combination therapy comprising a Clostridial toxin and a TEM reduces a symptom associated with a sensory disturbance disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a standard or low combination therapy comprising a Clostridial toxin and a TEM reduces a symptom associated with a sensory disturbance disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a standard or low combination therapy comprising a Clostridial toxin and a TEM reduces a symptom associated with a sensory disturbance disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a standard or low combination therapy comprising a Clostridial toxin and a TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a standard or low combination therapy comprising a Clostridial toxin and a TEM generally is in a Clostridial toxin: TEM molar ratio of about 1:1 to about 1:10,000. In other aspects of this embodiment, a therapeutically effective amount of a standard or low combination therapy comprising a Clostridial toxin and a TEM can be in a Clostridial toxin: TEM molar ratio of, e.g., about 1:1, about 1:2, about 1:5, about 1:10, about 1:25, about 1:50, about 1:75, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000. In yet other aspects of this embodiment, a therapeutically effective amount of standard or low combination therapy comprising a Clostridial toxin and a TEM can be in a Clostridial toxin: TEM molar ratio of, e.g., at least 1:1, at least 1:2, at least 1:5, at least 1:10, at least 1:25, at least 1:50, at least 1:75, at least 1:100, at least 1:200, at least 1:300, at least 1:400, at least 1:500, at least 1:600, at least 1:700, at least 1:800, at least 1:900, at least 1:1000, at least 1:2000, at least 1:3000, at least 1:4000, at least 1:5000, at least 1:6000, at least 1:7000, at least 1:8000, at least 1:9000, or at least 1:10,000. In still other aspects of this embodiment, a therapeutically effective amount of a standard or low combination therapy comprising a Clostridial toxin and a TEM can be in a Clostridial toxin: TEM molar ratio of between, e.g., about 1:1 to about 1:10,000, about 1:10 to about 1:10,000, about 1:100 to about 1:10,000, about 1:500 to about 1:10,000, about 1:1000 to about 1:10,000, about 1:5000 to about 1:10,000, about 1:1 to about 1:1000, about 1:10 to about 1:1000, about 1:100 to about 1:1000, about 1:250 to about 1:1000, about 1:500 to about 1:1000, about 1:750 to about 1:1000, about 1:1 to about 1:500, about 1:10 to about 1:500, about 1:50 to about 1:500, about 1:100 to about 1:500, about 1:250 to about 1:500, about 1:1 to about 1:100, about 1:10 to about 1:100, about 1:25 to about 1:100, about 1:50 to about 1:100, or about 1:75 to about 1:100.

In yet other aspects of this embodiment, a therapeutically effective amount of a standard combination therapy comprising a Clostridial toxin and a TEM generally is in a range of about 0.50 U to about 250 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

In yet other aspects of this embodiment, a therapeutically effective amount of a low combination therapy comprising a Clostridial toxin and a TEM generally is in a range of about 0.01 U to about 50 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

Dosing can be single dosage or cumulative (serial dosing), and can be readily determined by one skilled in the art. For instance, treatment of a sensory disturbance disorder may comprise a one-time administration of an effective dose of a composition disclosed herein. As a non-limiting example, an effective dose of a composition disclosed herein can be administered once to an individual, e.g., as a single injection or deposition at or near the site exhibiting a symptom of a sensory disturbance disorder. Alternatively, treatment of a sensory disturbance disorder may comprise multiple administrations of an effective dose of a composition disclosed herein carried out over a range of time periods, such as, e.g., daily, once every few days, weekly, monthly or yearly. As a non-limiting example, a composition disclosed herein can be administered once or twice yearly to an individual. The timing of administration can vary from individual to individual, depending upon such factors as the severity of an individual's symptoms. For example, an effective dose of a composition disclosed herein can be administered to an individual once a month for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

A composition disclosed herein can be administered to an individual using a variety of routes. Routes of administration suitable for a method of treating a sensory disturbance disorder as disclosed herein include both local and systemic administration. Local administration results in significantly more delivery of a composition to a specific location as compared to the entire body of the individual, whereas, systemic administration results in delivery of a composition to essentially the entire body of the individual. Routes of administration suitable for a method of treating a sensory disturbance disorder as disclosed herein also include both central and peripheral administration. Central administration results in delivery of a composition to essentially the central nervous system of an individual and includes, e.g., intrathecal administration, epidural administration as well as a cranial injection or implant. Peripheral administration results in delivery of a composition to essentially any area of an individual outside of the central nervous system and encompasses any route of administration other than direct administration to the spine or brain. The actual route of administration of a composition disclosed herein used can be determined by a person of ordinary skill in the art by taking into account factors, including, without limitation, the type of sensory disturbance disorder, the location of the sensory disturbance disorder, the cause of the sensory disturbance disorder, the severity of the sensory disturbance disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered systemically to an individual. In another embodiment, a composition disclosed herein is administered locally to an individual. In an aspect of this embodiment, a composition disclosed herein is administered to a nerve of an individual. In another aspect of this embodiment, a composition disclosed herein is administered to the area surrounding a nerve of an individual.

A composition disclosed herein can be administered to an individual using a variety of delivery mechanisms. The actual delivery mechanism used to administer a composition disclosed herein to an individual can be determined by a person of ordinary skill in the art by taking into account factors, including, without limitation, the type of sensory disturbance disorder, the location of the sensory disturbance disorder, the cause of the sensory disturbance disorder, the severity of the sensory disturbance disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered by injection. In aspects of this embodiment, administration of a composition disclosed herein is by, e.g., intramuscular injection, intraorgan injection, subdermal injection, dermal injection, intracranical injection, spinal injection, or injection into any other body area for the effective administration of a composition disclosed herein. In aspects of this embodiment, injection of a composition disclosed herein is to a nerve or into the area surrounding a nerve.

In another embodiment, a composition disclosed herein is administered by catheter. In aspects of this embodiment, administration of a composition disclosed herein is by, e.g., a catheter placed in an epidural space.

A composition disclosed herein as disclosed herein can also be administered to an individual in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A method of treating a sensory disturbance disorder in an individual, the method comprising the step of administering to the individual in need thereof a therapeutically effective amount of a composition including a Targeted Endopeptidase Modulator (TEM), wherein administration of the composition reduces a symptom of the sensory disturbance disorder, thereby treating the individual.
2. A use of a TEM in the manufacturing a medicament for treating a sensory disturbance disorder in an individual in need thereof.
3. A use of a TEM in the treatment of a sensory disturbance disorder in an individual in need thereof.
4. A method of treating a sensory disturbance disorder in an individual, the method comprising the step of administering to the individual in need thereof a therapeutically effective amount of a composition including a Clostridial neurotoxin and a TEM, wherein administration of the composition reduces a symptom of the sensory disturbance disorder, thereby treating the individual.
5. A use of a Clostridial neurotoxin and a TEM in the manufacturing a medicament for treating a sensory disturbance disorder in an individual in need thereof.
6. A use of a Clostridial neurotoxin and a TEM in the treatment of a sensory disturbance disorder in an individual in need thereof.
7. The embodiments of 1 to 6, wherein the TEM comprises a linear amino-to-carboxyl single polypeptide order of 1) a Clostridial toxin enzymatic domain, a Clostridial toxin translocation domain, a targeting domain, 2) a Clostridial toxin enzymatic domain, a targeting domain, a Clostridial toxin translocation domain, 3) a targeting domain, a Clostridial toxin translocation domain, and a Clostridial toxin enzymatic domain, 4) a targeting domain, a Clostridial toxin enzymatic domain, a Clostridial toxin translocation domain, 5) a Clostridial toxin translocation domain, a Clostridial toxin enzymatic domain and a targeting domain, or 6) a Clostridial toxin translocation domain, a targeting domain and a Clostridial toxin enzymatic domain.
8. The embodiments of 1 to 6, wherein the TEM comprises a linear amino-to-carboxyl single polypeptide order of 1) a Clostridial toxin enzymatic domain, an exogenous protease cleavage site, a Clostridial toxin translocation domain, a targeting domain, 2) a Clostridial toxin enzymatic domain, an exogenous protease cleavage site, a targeting domain, a Clostridial toxin translocation domain, 3) a targeting domain, a Clostridial toxin translocation domain, an exogenous protease cleavage site and a Clostridial toxin enzymatic domain, 4) a targeting domain, a Clostridial toxin enzymatic domain, an exogenous protease cleavage site, a Clostridial toxin translocation domain, 5) a Clostridial toxin translocation domain, an exogenous protease cleavage site, a Clostridial toxin enzymatic domain and a targeting domain, or 6) a Clostridial toxin translocation domain, an exogenous protease cleavage site, a targeting domain and a Clostridial toxin enzymatic domain.
9. The embodiments of 1 to 8, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
10. The embodiments of 1 to 9, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
11. The embodiments of 1 to 10, wherein the targeting domain is a sensory neuron targeting domain, a sympathetic neuron targeting domain, or a parasympathetic neuron targeting domain.
12. The embodiments of 1 to 10, wherein the targeting domain is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.
13. The embodiments of 8 to 12, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
14. The embodiments of 1 to 13, wherein the Clostridial neurotoxin is a BoNT/A, a BoNT/B, a BoNT/C1, a BoNT/D, a BoNT/E, a BoNT/F, a BoNT/G, a TeNT, a BaNT, a BuNT, or any combination thereof.
15. The embodiments of 1 to 14, wherein the sensory disturbance disorder is a sensory processing disorder, an auditory disturbance, an olfactory disturbance, a gustatory disturbance, a visual disturbance, a hallucination disorder, a nausea, a vomiting, or a gastrointestinal dysfunction.
16. The embodiment of 15, wherein the sensory processing disorder is a Type I, a sensory modulation disorder, a Type II, a sensory-based motor disorder, a Type III, a sensory discrimination disorder, an autism, or an attention-deficit hyperactivity disorder.
17. The embodiment of 15, wherein the auditory disturbance is an phonophobia, an auditory distortion, auditory dysfunction, or an auditory hallucination.
18. The embodiment of 15, wherein the olfactory disturbance is an osmophobia, an olfactory distortion, an olfactory dysfunction, a dysosmia, an anosmia, a hyposmia, a parosmia, a cacosmia, a troposmia, or an olfactory hallucination.
19. The embodiment of 15, wherein the gustatory disturbance is an ageusia, a hypogeusia, a hypergeusia, a dysgeusia, or a gustatory hallucination.
20. The embodiment of 15, wherein the visual disturbance is a photophobia, a visual distortion, or a visual hallucination.
21. The embodiment of 15, wherein the hallucination disorder is a chronoceptive hallucination, an equilibrioceptive hallucination, gustatory hallucination, nociceptive hallucination, an olfactory hallucination, a proprioceptive hallucination, a tactile hallucination, a thermoceptive hallucination, or a visual hallucination.
22. The embodiment of 15, wherein the nausea is an acute nausea, a short-term nausea, a persistent nausea, a chronic nausea, a recurring nausea, an unexplained nausea, a breakthrough nausea, or a refractory nausea.
23. The embodiment of 15, wherein the vomiting disorder is an acute vomiting, a short-term vomiting, a persistent vomiting, a chronic vomiting, a recurring vomiting, an unexplained vomiting, a breakthrough vomiting, a refractory vomiting, or a retching.
24. The embodiment of 15, wherein the gastrointestinal dysfunction is diarrhea or constipation.
25. The embodiments of 1 to 21, wherein the TEM is administered to an Arnold's nerve, a nerve from the vagal nerve complex, a nerve from the trigeminal nerve complex, or a nerve from the cervical nerve complex.
26. The embodiments of 1 to 25 further comprising administration of a suboptimal dose of a botulinum toxin.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions, methods or uses of treating a sensory disturbance disorder.

### Example 1

### Treating a Sensory Processing Disorder

A child experiences difficulty in processing and responding to sensory stimuli. After routine history and physical examination, a physician diagnosis the patient with an autism disorder involving abnormal sensory neuron activity and identifies the nerves and/or muscles involved in the condition. The boy is treated by injection of a composition comprising a TEM as disclosed in the present specification, targeting the Arnold's nerve in the external auditory canal. Alternatively, the boy may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement in processing and responding to sensory stimuli would be expected to continue.

A similar treatment regime can be used to treat any sensory processing disorder including 1) a Type I, a sensory modulation disorder; 2) a Type II, a sensory-based motor disorder; 3) a Type III, a sensory discrimination disorder; and 4) an attention-deficit hyperactivity disorder. Likewise, a similar therapeutic effect can be achieved with a suboptimal amount of any of the Clostridial toxins disclosed herein.

### Example 2

### Treating an Auditory Disturbance

A woman complains of being incredibly sensitive to sounds to a point where it interferes with her job performance. After routine history and physical examination, a physician diagnosis the patient with a phonophobia disorder involving abnormal sensory neuron activity and identifies the nerves and/or muscles involved in the condition. The woman is treated by injection of a composition comprising a TEM as disclosed in the present specification, targeting the Arnold's nerve in the external auditory canal. Alternatively, the woman may be treated by injecting a composition comprising a TEM and a suboptimal amount of a BoNT as disclosed in the present specification. The patient's condition would be expected to improve after about 2 weeks from treatment. At two and four month check-ups, improvement would be expected to continue. This decrease sensitivity to sound indicates a successful treatment with the composition comprising a TEM and a BoNT, such as BoNT/A, as disclosed in the present specification. Alternatively, the patient may be treated by injecting a composition comprising a BoNT.

A similar treatment regime can be used to treat any other auditory disturbance including an auditory distortion, auditory dysfunction, or an auditory hallucination. Likewise, a similar therapeutic effect can be achieved with a suboptimal amount of any of the Clostridial toxins disclosed herein.

### Example 3

### Treating an Olfactory Disturbance

A man complains being incredibly sensitive to certain odors to a point where it gives him a headache. After routine history and physical examination, a physician diagnosis the patient with an osmophobia disorder involving abnormal sensory neuron activity and identifies the nerves and/or muscles involved in the condition. The man is treated by injection of a composition comprising a TEM as disclosed in the present specification, targeting the dermatomal distribution of the nasociliary branch. Alternatively, the man may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This decreased sensitivity to odors indicates a successful treatment with the composition comprising a TEM.

A similar treatment regime can be used to treat any other olfactory disturbance disorder including 1) an olfactory distortion; 2) an olfactory dysfunction; 3) a dysosmia; 4) an anosmia; 5) a hyposmia; 6) a parosmia; 7) a cacosmia; 8) a troposmia; and 9) olfactory hallucination. Likewise, a similar therapeutic effect can be achieved with a suboptimal amount of any of the Clostridial toxins disclosed herein.

### Example 4

### Treating a Gustatory Disturbance

A man complains of not being able to taste anything. After routine history and physical examination, a physician diagnosis the patient with an ageusia disorder involving abnormal sensory neuron activity and identifies the nerves and/or muscles involved in the condition. The man is treated by injection of a composition comprising a TEM as disclosed in the present specification, targeting the branches of the vagal nerve. Alternatively, the man may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This increased sensitivity to taste indicates a successful treatment with the composition comprising a TEM.

A similar treatment regime can be used to treat any other gustatory disturbance disorder including an ageusia, a hypogeusia, a hypergeusia, a dysgeusia, and a gustatory hallucination. Likewise, a similar therapeutic effect can be achieved with a suboptimal amount of any of the Clostridial toxins disclosed herein.

### Example 5

### Treating a Visual Disturbance

A woman complains of being incredibly sensitive to light to a point where it interferes with her job performance. After routine history and physical examination, a physician diagnosis the patient with a photophobia disorder involving abnormal sensory neuron activity and identifies the nerves and/or muscles involved in the condition. The woman is treated by injection of a composition comprising a TEM as disclosed in the present specification, targeting the Arnold's nerve in the external auditory canal. Alternatively, the woman may be treated by injecting a composition comprising a TEM and a suboptimal amount of a BoNT, such as BoNT/A, as disclosed in the present specification. Alternatively, the patient may be treated by injecting a composition comprising a BoNT. The patient's condition is monitored and after about 2 days from treatment, the woman indicates she has decreased sensitivity to light. At two and four month check-ups, the woman indicates that she is still experiencing decreased sensitivity to sound. This decrease sensitivity to light indicates a successful treatment with the composition comprising a TEM and a BoNT, such as BoNT/A, as disclosed in the present specification. Alternatively, the patient may be treated by injecting a composition comprising a BoNT.

A similar treatment regime can be used to treat any other visual disturbance including a visual distortion and a visual hallucination. Likewise, a similar therapeutic effect can be achieved with a suboptimal amount of any of the Clostridial toxins disclosed herein.

### Example 6

### Treating a Hallucination

A man complains about hearing things even though there is nothing making that sound. After routine history and physical examination, a physician diagnosis the patient with an auditory hallucination disorder involving abnormal sensory neuron activity and identifies the nerves and/or muscles involved in the condition. The man is treated by injection of a composition comprising a TEM as disclosed in the present specification, targeting the Arnold's nerve in the external auditory canal. Alternatively, the man may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This decrease in auditory hallucinations indicates a successful treatment with the composition comprising a TEM.

A similar treatment regime can be used to treat any other hallucination disorder including 1) a chronoceptive hallucination; 2) an equilibrioceptive hallucination; 3) gustatory hallucination; 4) nociceptive hallucination; 5) an olfactory hallucination; 6) a proprioceptive hallucination; 7) a tactile hallucination; 8) a thermoceptive hallucination; and 9) a visual hallucination. Likewise, a similar therapeutic effect can be achieved with a suboptimal amount of any of the Clostridial toxins disclosed herein.

### Example 7

### Treatment of a Nausea

A man complains about experiencing discomfort and queasiness in the upper stomach. After routine history and physical examination, a physician diagnosis the patient with a nausea disorder involving abnormal sensory neuron activity and identifies the nerves and/or muscles involved in the condition. The man is treated by injection of a composition comprising a TEM as disclosed in the present specification, targeting the Arnold's nerve in the external auditory canal. Alternatively, the man may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This decrease in nausea indicates a successful treatment with the composition comprising a TEM.

A similar treatment regime can be used to treat any seizure disorder including 1) an acute nausea; 2) a short-term nausea; 3) a persistent nausea; 4) a chronic nausea; 5) a recurring nausea; 6) an unexplained nausea; 7) a breakthrough nausea; and 8) a refractory nausea. Likewise, a similar therapeutic effect can be achieved with a suboptimal amount of any of the Clostridial toxins disclosed herein.

### Example 8

### Treatment of a Vomiting

A woman complains of vomiting. After routine history and physical examination, a physician diagnosis the patient with a vomiting disorder involving abnormal sensory neuron activity and identifies the nerves and/or muscles involved in the condition. The woman is treated by injection of a composition comprising a TEM as disclosed in the present specification, targeting the Arnold's nerve in the external auditory canal. Alternatively, the woman may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This decrease in vomiting episodes indicates a successful treatment with the composition comprising a TEM and a BoNT, such as BoNT/A, as disclosed in the present specification. Alternatively, the patient may be treated by injecting a composition comprising a BoNT.

A similar treatment regime can be used to treat any vomiting disorder including 1) an acute vomiting; 2) a short-term vomiting; 3) a persistent vomiting; 4) a chronic vomiting; 5) a recurring vomiting; 6) an unexplained vomiting; 7) a breakthrough vomiting; 8) a refractory vomiting; and 9) a retching. Likewise, a similar therapeutic effect can be achieved with a suboptimal amount of any of the Clostridial toxins disclosed herein.

### Example 9

### Treating a Gastrointestinal Dysfunction

A woman complains of diarrhea. After routine history and physical examination, a physician diagnosis the patient with a diarrhea disorder involving abnormal sensory neuron activity and identifies the nerves and/or muscles involved in the condition. The woman is treated by injection of a composition comprising a TEM as disclosed in the present specification, targeting the Arnold's nerve in the external auditory canal. Alternatively, the woman may be treated by injecting a composition comprising a TEM and a suboptimal amount of a BoNT, such as BoNT/A, as disclosed in the present specification. Alternatively, the patient may be treated by injecting a composition comprising a BoNT. The patient's condition would be expected to improve after about 2 weeks from treatment. At two and four month check-ups, improvement would be expected to continue. This decrease in diarrhea episodes indicates a successful treatment with the composition comprising a TEM and a BoNT, such as BoNT/A, as disclosed in the present specification. Alternatively, the patient may be treated by injecting a composition comprising a BoNT.

A similar treatment regime can be used to treat any other gastrointestinal dysfunction disorder including constipation. Likewise, a similar therapeutic effect can be achieved with a suboptimal amount of any of the Clostridial toxins disclosed herein.

### Example 10

### Treating Nausea and Sensitivity to Light and Sound

A 20 year old woman, with migraine since 10 years of age and fibromyalgia since 14 years of age, complains of nausea and sensitivity to light and sound. Headaches had escalated to daily by late teens. Patient tried numerous oral preventive medications without benefit and had failed on Topamax®. Headache days almost daily at baseline. Botox® treatment using the PREEMPT paradigm (U.S. Ser. No. 13/075,485, filed Mar. 30, 2011) started 3 years previous, repeating every 3 months. Initial dose 100 units and then increased to 200 units. After ten treatments, headache days decreased to 70/90, with MIDAS 115 and intensity 7/10. PHQ 9 score 9, consistent with mild depression, on Lamictal® and Cymbalta® for depression. Botox decreased the associated symptoms of migraine such as sensitivity to light and sound, also nausea. As a result the disability was lessened. Intensity would have been a 10/10 before treatment. The reduction in intensity is due to the reduction in the associated symptoms. Before Botox® these intense headaches occurred about once every 2 weeks and decreased with treatment to once every 2 months. Nausea is not present after treatment.

Patient could also be treated with a composition comprising a TEM as disclosed in the present specification. A TEM injection could target the Arnold's nerve in the external auditory canal or the sphenopalatine ganglion. Alternatively, the woman could be treated by injecting a composition comprising a TEM and a suboptimal amount of a BoNT as disclosed in the present specification. The patient's condition would be expected to improve after about 2 weeks from treatment. At two and four month check-ups, improvement would be expected to continue.

### CONCLUSION

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a sensory disturbance disorder in an individual, the method comprising the step of administering to the individual in need thereof a therapeutically effective amount of a composition including a TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain, and wherein administration of the composition reduces a symptom of the sensory disturbance disorder, thereby treating the individual.

2. The method of claim 1, wherein the TEM comprises a linear amino-to-carboxyl single polypeptide order of 1) the Clostridial toxin enzymatic domain, the Clostridial toxin translocation domain, the targeting domain, 2) the Clostridial toxin enzymatic domain, the targeting domain, the Clostridial toxin translocation domain, 3) the targeting domain, the Clostridial toxin translocation domain, and the Clostridial toxin enzymatic domain, 4) the targeting domain, the Clostridial toxin enzymatic domain, the Clostridial toxin translocation domain, 5) the Clostridial toxin translocation domain, the Clostridial toxin enzymatic domain and the targeting domain, or 6) the Clostridial toxin translocation domain, the targeting domain and the Clostridial toxin enzymatic domain.

3. The method of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

4. The method of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

5. The method of claim 1, wherein the sensory disturbance disorder is a sensory processing disorder, an auditory disturbance, an olfactory disturbance, a gustatory disturbance, a visual disturbance, a hallucination disorder, a nausea, a vomiting, or a gastrointestinal dysfunction.

6. The method of claim 5, wherein the sensory processing disorder is a Type I, a sensory modulation disorder, a Type II, a sensory-based motor disorder, a Type III, a sensory discrimination disorder, an autism, or an attention-deficit hyperactivity disorder.

7. The method of claim 5, wherein the auditory disturbance is an phonophobia, an auditory distortion, auditory dysfunction, or an auditory hallucination.

8. The method of claim 5, wherein the olfactory disturbance is an osmophobia, an olfactory distortion, an olfactory dysfunction, a dysosmia, an anosmia, a hyposmia, a parosmia, a cacosmia, a troposmia, or an olfactory hallucination.

9. The method of claim 5, wherein the gustatory disturbance is an ageusia, a hypogeusia, a hypergeusia, a dysgeusia, or a gustatory hallucination.

10. The method of claim 5, wherein the visual disturbance is a photophobia, a visual distortion, or a visual hallucination.

11. The method of claim 5, wherein the hallucination disorder is a chronoceptive hallucination, an equilibrioceptive hallucination, gustatory hallucination, nociceptive hallucination, an olfactory hallucination, a proprioceptive hallucination, a tactile hallucination, a thermoceptive hallucination, or a visual hallucination.

12. The method of claim 5, wherein the nausea is an acute nausea, a short-term nausea, a persistent nausea, a chronic nausea, a recurring nausea, an unexplained nausea, a breakthrough nausea, or a refractory nausea.

13. The method of claim 5, wherein the vomiting disorder is an acute vomiting, a short-term vomiting, a persistent vomiting, a chronic vomiting, a recurring vomiting, an unexplained vomiting, a breakthrough vomiting, a refractory vomiting, or a retching.

14. The method of claim 5, wherein the gastrointestinal dysfunction is diarrhea or constipation.

15. The method of claim 1, wherein the TEM is administered to an Arnold's nerve, a nerve from the vagal nerve complex, a nerve from the trigeminal nerve complex, or a nerve from the cervical nerve complex.

16. The method of claim 1, further comprising administration of a sub-optimal amount of a botulinum toxin.

17. A use of a TEM in the treatment of a sensory disturbance disorder in an individual in need thereof, wherein the TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain.

18. A use of a TEM in the treatment of a sensory disturbance disorder in an individual in need thereof, the use comprising the step of administering to the individual a therapeutically effective amount of the composition, wherein the TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain.
